(19) United States
(12) Patent Application Publication
(10) Pub. No.: US 2026/0191786 A1
(43) Pub. Date: Jul. 9, 2026

(54) SOLID DOSAGE FORMS WITH HIGH ACTIVE AGENT LOADING

(71) Applicant: Lonza Sales AG, Basel (CH)

(72) Inventors: Deanna Mudie, Bend, OR (US); Michael M. Morgen, Bend, OR (US); Kimberly Shepard, Bend, OR (US)

(73) Assignee: Lonza Sales AG, Basel (CH)

(21) Appl. No.: 19/372,787

(22) Filed: Oct. 29, 2025

Related U.S. Application Data
(63) Continuation of application No. 17/054,895, filed on May 9, 2019, now abandoned.
(60) Provisional application No. 62/671,341, filed on May 14, 2018.

(51) Int. Cl. A61K 9/20; A61K 9/16

(52) U.S. Cl. CPC A61K 9/2027; A61K 9/1635; A61K 9/1652; A61K 9/2054

(57) ABSTRACT

This disclosure concerns oral pharmaceutical compositions comprising a solid dosage form (SDF). The SDF comprises (i) a solid amorphous dispersion (SAD) comprising a poorly water soluble active agent and a matrix material comprising poly[(methyl methacrylate)-co-(methacrylic acid)](PMMAMA), and (ii) a concentration-sustaining polymer (CSP), wherein the CSP is not dispersed in the SAD, and the SAD is at least 35 wt % of the SDF. The SAD and CSP together may be at least 50 wt % of the SDF. The SDF may be, for example, a tablet, a caplet, or a capsule.

### CROSS-REFERENCE TO RELATED APPLICATIONS

This application is a continuation of U.S. patent application Ser. No. 17/054,895, filed Nov. 12, 2020, which is the U.S. National Stage of International Application No. PCT/IB2019/053836, filed May 9, 2019, which was published in English under PCT Article 21(2), which in turn claims the benefit of the earlier filing date of U.S. Provisional Application No. 62/671,341, filed May 14, 2018, which are incorporated by reference in their entirety herein.

### FIELD

This disclosure concerns solid dosage forms comprising (i) a solid amorphous dispersion including an active agent and a dispersion polymer, and (ii) a concentration-sustaining polymer.

### BACKGROUND

Solid amorphous dispersions (SADs)—including spray-dried dispersions (SDDs), spray-layered dispersions (SLDs) and amorphous dispersions made by hot melt extrusion (HME)—may increase the absorption of low-solubility active agents from the gastrointestinal (GI) tract by increasing dissolution rate, maximizing dissolved active agent concentration, and/or sustaining high active agent concentrations. However, for many SADs, it is difficult to achieve these objectives while also achieving a high active agent loading in the solid dosage form (SDF). Often, the active agent loading is limited by physical stability, especially for drugs having a low glass transition temperature (Tg). Also, regardless of physical stability limitations, SDFs incorporating a high proportion of a binary SDD including an active agent and a concentration-sustaining polymer (CSP) often disintegrate and/or dissolve unacceptably slowly. In some cases, a compressed tablet incorporating a high level of a CSP may gel upon wetting, forming a hydrated monolithic mass that is resistant to disintegration or dissolution. The problem is exacerbated when the SDD has a high loading (e.g., >50 wt %) of a hydrophobic, poorly water soluble active agent that may have a high solubility in the wet CSP upon exposure to aqueous media.

### SUMMARY

Oral pharmaceutical compositions comprising a solid dosage form (SDF) are disclosed. The SDF comprises (i) a solid amorphous dispersion (SAD) comprising a poorly water soluble active agent and a matrix material comprising poly[(methyl methacrylate)-co-(methacrylic acid)](PMMAMA), the PMMAMA having a glass transition temperature T_{g}≥135° C. at <5% relative humidity, and (ii) a concentration-sustaining polymer (CSP). The CSP is not PMMAMA and is not dispersed in the SAD. The SAD is at least 35 wt % of the SDF. In some embodiments, the CSP comprises hydroxypropyl methylcellulose acetate succinate (HPMCAS), hydroxypropyl methylcellulose (HPMC), poly(vinylpyrrolidone-co-vinyl acetate) (PVPVA), carboxymethyl ethylcellulose (CMEC), or a combination thereof. In any or all of the above embodiments, the poorly water soluble active agent may have a melting temperature Tₘ to glass transition temperature T_{g} ratio≥1.3 and a Log P<10.

In any or all of the above embodiments, (i) the SAD may have an active agent loading of at least 35 wt %, (ii) at least 95% of the SAD particles may have an aspect ratio <10, (iii) the PMMAMA may have a free carboxyl group to ester group ratio of from 1:0.8 to 1:2.2, or (iv) any combination of (i), (ii), and (iii). In any or all of the above embodiments, (i) the SAD may be at least 40 wt %, at least 50 wt %, at least 60 wt %, at least 70 wt %, or even at least 75 wt % of the SDF; (ii) the CSP may be at least 5 wt % of the SDF, at least 10 wt % of the SDF, at least 20 wt % of the SDF, or even at least 25 wt % of the SDF; (iii) the SAD and the CSP together may be at least 50 wt % of the SDF, at least 60 wt % of the SDF, at least 70 wt % of the SDF, at least 80 wt % of the SDF, or even at least 90 wt % of the SDF; (iv) a ratio of the CSP to the active agent may be from 0.4:1 to 5:1, 0.5:1 to 3:1, or even 0.8:1 to 2:1; or (iv) any combination of (i), (ii), (iii), and (iv).

In any or all of the above embodiments, the SDF may comprise a granular blend comprising particles of the SAD and particles of the CSP, or an intragranular blend wherein individual granules comprise SAD particles and CSP particles. In some embodiments, at least some of the individual granules of the intragranular blend comprise SAD particles, CSP particles, and one or more intragranular excipients. The SDF may further comprise one or more extragranular excipients.

In one embodiment, the SDF is a compressed tablet or caplet, wherein the SAD and CSP are blended and compressed to form the tablet or caplet. In another embodiment, the SDF is a compressed tablet or caplet comprising compressed SAD particles and an outer coating comprising the CSP. In yet another embodiment, the SDF is a capsule comprising a capsule shell and a fill comprising the SAD and the CSP. In still another embodiment, the SDF is a capsule comprising a capsule shell comprising the CSP and a fill comprising the SAD.

The foregoing and other objects, features, and advantages of the invention will become more apparent from the following detailed description, which proceeds with reference to the accompanying figures.

### BRIEF DESCRIPTION OF THE DRAWINGS

FIG. 1 is a table showing formulations of several erlotinib tablet compositions.

FIG. 2 is a table showing excipients used in the tablet compositions of FIG. 1.

FIG. 3 is a graph showing dissolution performance of the tablet compositions of FIG. 1.

FIG. 4 is a graph showing dissolution performance of two 300 mg erlotinib tablets wherein a concentration-sustaining polymer is (i) included within an intragranular blend with a spray-dried amorphous dispersion comprising an active agent and dispersion polymer, or (ii) external to the intragranular blend.

FIG. 5 is a graph showing dissolution performance of two 400 mg erlotinib tablets wherein a concentration-sustaining polymer is (i) included within an intragranular blend with a spray-dried amorphous dispersion comprising an active agent and dispersion polymer, or (ii) external to the intragranular blend.

FIG. 6 is a graph showing the glass transition temperature T_{g} of PMMAMA-based and HPMCAS-H-based SDDs with varying drug loadings as a function of relative humidity (RH).

FIG. 7 is a table showing formulations of several erlotinib tablet compositions.

FIG. 8 is a graph showing dissolution performance of two erlotinib tablet compositions of FIG. 7 wherein the dispersion polymer is Eudragit® L100 (PMMAMA) polymer compared to a benchmark composition.

FIG. 9 is a graph showing dissolution performance of two erlotinib tablet compositions of FIG. 7 wherein the dispersion polymer is Eudragit® S100 (PMMAMA) polymer compared to a benchmark composition.

FIG. 10 is a graph showing the glass transition temperature (T_{g}) of SDDs with Eudragit® 5100 (PMMAMA) polymer or Eudragit® L100 (PMMAMA) polymer at a drug loading of 65 wt % erlotinib compared to a 35 wt % erlotinib in HPMCAS-H SAD as a function of relative humidity (RH).

FIG. 11 is a table showing formulations of several posaconazole tablet compositions.

FIG. 12 is a table showing excipients used in the tablet compositions of FIG. 11.

FIG. 13 is a graph showing dissolution performance of the tablet compositions of FIG. 11.

FIG. 14 is a graph showing the T_{g} of SDDs with Eudragit® L100 (PMMAMA) polymer at drug loadings of 50-85 wt % posaconazole compared to 35-75 wt % posaconazole in HPMCAS-H SDDs as a function of RH.

### DETAILED DESCRIPTION

This disclosure concerns oral pharmaceutical compositions, particularly oral compositions comprising a solid dosage form (SDF), the SDF comprising a SAD. Some embodiments of the disclosed oral pharmaceutical compositions exhibit a) good physical stability (e.g., with respect to active agent phase separation/crystallization), b) rapid dissolution rate, c) sustainment of supersaturated active agent, d) high active agent loading, or any combination thereof. Advantageously, certain embodiments of the oral pharmaceutical compositions provide improved oral bioavailability of low-soluble active agents using a minimum number of dosage units.

### I. DEFINITIONS AND ABBREVIATIONS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The indefinite article “a” or “an” thus usually means “at least one.” The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” The term “about” as used in the disclosure of numerical ranges indicates that deviation from the stated value is acceptable to the extent that the deviation is the result of measurement variability and/or yields a product of the same or similar properties. Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 1997 (ISBN 0-471-29205-2). In order to facilitate review of the various embodiments of the disclosure, the following explanations of specific terms are provided:

Active: As used herein, the term “active ingredient,” “active substance,” “active component,” “active pharmaceutical ingredient” and “active agent” have the same meaning as a component which exerts a desired physiological effect on a mammal, including but not limited to humans.

Amorphous: Non-crystalline. Amorphous solids lack a definite crystalline structure and a well-defined melting point.

Aspect ratio: As used herein with respect to particles, the term “aspect ratio” refers to the ratio of length to width. The length is defined as the maximum straight-line distance between two points on the particle. The width is taken at the midpoint of the length, on a line perpendicular to the line which defines the length. If the particle twists or folds back over itself, then a contour length (i.e., length at maximum physical extension) measurement is used. A particle's aspect ratio may be measured by optical or electron microscopy techniques, e.g., by scanning electron microscopy whereby individual particles may be visualized at magnification and measured. ImageJ open-source software may be used to automate counting of particles with a low aspect ratio, e.g., an aspect ratio <10.

Concentration-sustaining polymer (CSP): A polymer that provides an initially enhanced dissolved concentration of an active agent in an in vivo or in vitro use environment (e.g., a subject's gastrointestinal tract, simulated intestinal fluid, model fasted duodenal solution, and the like) relative to a benchmark composition that does not include the CSP and maintains a greater dissolved concentration of the active agent over an extended period of time (e.g., at least 30 minutes, such as for 30-90 minutes) relative to the benchmark composition in the same use environment. The dissolved concentration can be assessed by any suitable method. For example, an in vitro dissolved concentration may be determined by UV-visible spectroscopy at a wavelength absorbed by the active agent. A calibration curve using known concentrations of the active agent is prepared for comparison.

Dispersion: A system in which particles, e.g., particles of an active agent, are distributed within a continuous phase of a different composition. A solid dispersion is a system in which at least one solid component is distributed throughout another solid component. A molecular dispersion is a system in which at least one component is homogeneously or substantially homogeneously dispersed on a molecular level throughout another component.

Excipient: A physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Examples of excipients include but are not limited to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Extragranular: External to granules. For example, granules mixed with a polymer or excipients that are not part of the granules.

Glass transition temperature, T_{g}: The temperature at which a material transitions from a supercooled liquid to a glass. T_{g} can be determined, for example, by differential scanning calorimetry (DSC). DSC measures the difference in the amount of heat required to raise the temperature of a sample and a reference as a function of temperature. During a phase transition, such as a change from an amorphous state to a crystalline state, the amount of heat required changes. For a solid that has no crystalline components, a single glass transition temperature indicates that the solid is homogeneous or a molecular dispersion. In general, when a glass is tested by increasing the temperature of the sample at a constant rate, typically 1 to 10° C./min, a relatively sharp increase in heat capacity will be observed in the vicinity of the T_{g}. T_{g} can also be measured by a dynamic mechanical analyzer (DMA), a dilatometer, or by dielectric spectroscopy. T_{g} values measured by each technique may vary, but generally fall within 10-30° C. of one another. For example, the T_{g} measured by DMA is often 10-30° C. higher than the T_{g} measured by DSC.

Granular: Granular particles have an average diameter of 100-600 μm. As used herein, “average diameter” means the mathematical average diameter of a plurality of granules.

Granular blend: A plurality of granules comprising two or more components. Each granule may include one component or more than one component.

Intragranular blend: A plurality of granules, each granule comprising two or more components, e.g., each granule comprising active agent and polymer.

Loading: The term “loading” as used herein refers to a percentage by weight of an active agent in a solid amorphous dispersion, spray-dried dispersion, or solid dosage form.

Log P: The Log P value of an active agent is defined as the base 10 logarithm of the ratio of (1) the active agent concentration in an octanol phase to (2) the active agent concentration in a water phase when the two phases are in equilibrium with each other, is a widely accepted measure of lipophilicity. The Log P value may be measured experimentally or calculated using methods known in the art. The Log P value may be estimated experimentally by determining the ratio of the drug solubility in octanol to the drug solubility in water. When using a calculated value for the Log P value, the highest value calculated using any generally accepted method for calculating Log P is used. Calculated Log P values are often referred to by the calculation method, such as Clog P, Alog P, and Mlog P. The Log P value may also be estimated using fragmentation methods, such as Crippen's fragmentation method (J. Chem. Inf Comput. Sci., 27, 21 (1987)); Viswanadhan's fragmentation method (J. Chem. Inf. Comput. Sci., 29,163 (1989)); or Broto's fragmentation method (Eur. J. Med. Chem.-Chim. Theor. 19, 71 (1984). In some embodiments, the Log P value is calculated by using the average value estimated using Crippen's, Viswanadhan's, and Broto's fragmentation methods.

Matrix: As used herein, the term “matrix” or “matrix material” refers to a polymeric material in which an active agent is mixed or dispersed.

Melting temperature, Tₘ: The temperature at which a compound changes state from solid to liquid at atmospheric pressure. Tₘ can be determined, for example, by differential scanning calorimetry (DSC). DSC measures the difference in the amount of heat required to raise the temperature of a sample and a reference as a function of temperature. During a phase transition, such as a change from a solid state to a liquid state, the amount of heat required changes. Alternatively, Tₘ can be determined with a basic melting point apparatus including an oil bath with a transparent window and a magnifier. Several grains of solid are placed in a thin glass tube and partially immersed in the oil bath. The oil bath is heated and stirred, and the temperature at which the grains melt can be observed by manual or automated detection.

PMMAMA: Poly[(methyl methacrylate)-co-(methacrylic acid)].

SDD: Spray-dried dispersion.

SDF: Solid dosage form.

Solid amorphous dispersion (SAD): A solid dispersion including an active agent dispersed in a polymer, wherein the active agent is amorphous or substantially (at least 80 wt %) amorphous. A SAD is often prepared by a spray-drying process. Unless otherwise specified, the terms SAD and spray-dried dispersion (SDD) are used interchangeably in this disclosure.

Supersaturated: A state in which a solution includes a dissolved solute at a greater concentration than the equilibrium dissolved concentration of the solute in the solvent at a given temperature.

### II. ORAL PHARMACEUTICAL COMPOSITIONS

Embodiments of the disclosed oral pharmaceutical compositions comprise a solid dosage form (SDF) comprising (i) a SAD comprising a poorly water soluble active agent in amorphous or substantially amorphous (i.e., at least 80 wt % amorphous) form and a matrix material comprising one or more dispersion polymers, and (ii) one or more concentration-sustaining polymers (CSPs), wherein the one or more CSPs are not dispersed within the SAD, and the dispersion polymer and CSPs are different polymers. In some embodiments, the SDF has an active agent loading that is at least 50% higher than the active agent loading in a reference SDF comprising a SAD comprising the poorly water soluble active agent in amorphous form and the CSP polymer alone, the matrix dispersion polymer alone, or a mixture of the two polymers. Advantageously, certain embodiments of the disclosed SDFs also provide rapid disintegration to obtain supersaturated dissolved active agent concentrations and/or sustainment of supersaturated active agent concentrations for a prolonged period of time.

The foregoing benefits, among others, are achieved by strategically distributing functionality across the entire SDF. Conventional SDFs comprise an optimized SAD that is then incorporated into a dosage form without doing harm to the performance. A conventional SDF typically comprises an optimized SAD, or a physical mixture of an active agent and one or more polymers, that is combined with excipients to form the SDF. In contrast, embodiments of the disclosed SDFs comprise an SAD and a CSP that are combined into a SDF. By distributing the functionality (e.g., rapid disintegration with concentration sustainment) across the entire SDF, an SDF with a higher active agent loading and greater physical stability can be provided.

### Solid Amorphous Dispersion

The solid amorphous dispersion comprises a poorly water soluble active agent in amorphous or substantially amorphous (i.e., at least 80 wt % amorphous) form and a matrix material comprising one or more dispersion polymers. The SAD may be a spray-dried dispersion.

A poorly water soluble active agent has low aqueous solubility in an amorphous state and/or a crystalline state, i.e., an aqueous solubility ≤1 mg/mL, over at least a portion of a physiologically relevant pH range of 1-8. In some embodiments, the poorly water soluble active agent has an aqueous solubility of ≤1 mg/mL or ≤0.1 mg/mL, such as an aqueous solubility of 0.0001-1 mg/mL or 0.0001-0.1 mg/mL over at least a portion of the physiologically relevant pH range of 1-8. In any or all of the foregoing embodiments, the active agent may be more soluble in an amorphous state than in a crystalline state. In some embodiments, the active agent has a high ratio of amorphous to crystalline solubility, such as an amorphous solubility to crystalline solubility ratio >5, >10, or even >20.

A driving force for crystallization is a ratio of the melting temperature (Tₘ) of the poorly water soluble active agent to its glass transition temperature (T_{g}). Compounds with high melting points have a strong tendency to crystallize, and compounds with low T_{g} values have a low kinetic barrier for molecular diffusion. Thus, the Tₘ/T_{g} ratio (K/K) provides an indication of a compound's tendency to crystallize. Compounds with a higher ratio are more likely to crystallize. In any or all of the above embodiments, the active agent may have a Tₘ/T_{g} ratio ≥1.2, such as a Tₘ/T_{g} ratio ≥1.3, ≥1.35, ≥1.4, ≥1.5, or ≥1.6, such as a Tₘ/T_{g} ratio of 1.2-2.0, 1.3-1.6, 1.35-1.6, or 1.4-1.6.

Log P is a measure of the poorly water soluble active agent's lipophilicity. In any or all of the above embodiments, the poorly water soluble active agent may have a Log P≥2 and/or ≤10, such as a Log P within a range of 1-10, 2-10, 3-10, 4-10, or 5-10.

In some embodiments, the poorly water soluble active agent is a “rapid crystallizer.” In some embodiments, a rapid crystallizer has a Tₘ/T_{g} ratio ≥1.3 such as a Tₘ/T_{g} ratio ≥1.35 or ≥1.4, and a Log P within a range of 1-10. In certain embodiments, a rapid crystallizer has a Tₘ/T_{g} ratio within a range of 1.4-2.0 or 1.4-1.6, and a Log P within a range of 1-7, 2-7, 3-7, 4-7, or 5-7.

Non-limiting examples of active agents according to the disclosure include but are not limited to poorly water soluble drugs, dietary supplements, such as vitamins or provitamins A, B, C, D, E, PP and their esters, carotenoids, anti-radical substances, hydroxyacids, antiseptics, molecules acting on pigmentation or inflammation, biological extracts, antioxidants, cells and cell organelles, antibiotics, macrolides, antifungals, itraconazole, ketoconazole, antiparasitics, antimalarials, adsorbents, hormones and derivatives thereof, nicotine, antihistamines, steroid and non-steroid anti-inflammatories, ibuprofen, naproxen, cortisone and derivatives thereof, anti-allergy agents, antihistamines, analgesics, local anesthetics, antivirals, antibodies and molecules acting on the immune system, cytostatics and anticancer agents, hypolipidemics, vasodilators, vasoconstrictors, inhibitors of angiotensin-converting enzyme and phosphodiesterase, fenofibrate and derivatives thereof, statins, nitrate derivatives and anti-anginals, beta-blockers, calcium inhibitors, anti-diuretics and diuretics, bronchodilators, opiates and derivatives thereof, barbiturates, benzodiazepines, molecules acting on the central nervous system, nucleic acids, peptides, anthracenic compounds, paraffin oil, polyethylene glycol, mineral salts, antispasmodics, gastric anti-secretory agents, clay gastric dressings and polyvinylpyrrolidone, aluminum salts, calcium carbonates, magnesium carbonates, starch, derivatives of benzimidazole, and combinations of the foregoing. Orally disintegrating tablets in certain embodiments of the instant disclosure may also comprise a glucuronidation inhibitor, for example, piperine.

Non-limiting exemplary active ingredients according to the present disclosure include dextromethorphan, erlotinib, fexofenadine, guaifenesin, loratadine, sildenafil, vardenafil, tadafil, olanzapine, risperidone, famotidine, loperamide, zolmitriptan, ondansetron, cetirizine, desloratadine, rizatriptan, piroxicam, paracetamol (acetaminophen), phloroglucinol, nicergoline, metopimazine, dihydroergotamine, mirtazapine, clozapine, prednisolone, levodopa, carbidopa, lamotrigine, ibuprofen, oxycodone, diphenhydramine, ramosetron, tramadol, zolpidem, fluoxetine, hyoscyamine, and combinations thereof. Placebo drug products are also within the scope of the instant disclosure and may be considered as an “active agent” in certain embodiments of the disclosed compositions.

A solid amorphous dispersion (SAD) is formed with the poorly water soluble active agent and a matrix material, i.e., a dispersion polymer in which the active agent is dispersed. In some embodiments, the active agent is homogeneously or substantially homogeneously dispersed throughout the dispersion polymer. In certain embodiments, the SAD is a molecular dispersion of the active agent and the dispersion polymer.

In some embodiments, the dispersion polymer has a T_{g}≥135° C. at <5% relative humidity (RH), such as a T_{g} of 135-200° C. at 5% RH. In any or all of the above embodiments, the dispersion polymer may have an acid content of ≥0.2 mol/100 g (≥2 mmol/g). The acid content refers to the number moles of acidic groups (e.g., ionizable protonated groups) per unit mass of the polymer. In some embodiments, the dispersion polymer has an acid content ≥0.3 mol/100 g, ≥0.4 mol/100 g, or ≥0.5 mol/100 g. In some embodiments, the dispersion polymer is a polymer comprising ionizable carboxy groups. The dispersion polymer is at least somewhat hydrophobic at low pH (e.g., pH<4.5) but becomes aqueous soluble when the carboxy groups are ionized at higher pH (e.g., >5.5). Dispersion polymers with these characteristics exhibit a low tendency to form a gel at a gastric pH of ~2, and readily dissolve at the higher pH of the intestine. Thus, the dispersion polymer may be an enteric polymer.

In any or all of the above embodiments, the matrix material, or dispersion polymer, may comprise poly[(methyl methacrylate)-co-(methacrylic acid)](PMMAMA). In some embodiments, the PMMAMA has a glass transition temperature (T_{g})≥135° C. at <5% relative humidity, such as a T_{g} within a range of 135-200° C. or 135-190° C. at <5% RH. In certain embodiments, the PMMAMA has a free carboxyl group to ester group ratio of from 1:0.8 to 1:2.2, providing 2.5-7 mmol acid/gram. PMMAMA is soluble in the intestinal tract, e.g., at a pH≥6. In one embodiment, the free carboxyl group to ester group ratio is from 1:0.8 to 1:1.2 or from 1:0.9 to 1:1.1. In an independent embodiment, the free carboxyl group to ester group ratio is from 1:1.8 to 1:2.2 or from 1:1.9 to 1:2.1. The PMMAMA may be a commercially available polymer sold under the tradenames Eudragit® L100 having a free carboxyl group to ester group ratio of approximately 1:1 and an acid content of 5.6 mmol acid/gram, or Eudragit® S100 having a free carboxyl group to ester group ratio of approximately 1:2 and an acid content of 3.5 mmol acid/gram (Evonik Nutrition & Care GmbH, Essen, Germany). The Eudragit® L100 and S100 polymers include ~0.3 wt % sodium lauryl sulfate.

The glass transition temperature of a SAD may be estimated to be a weighted average of the T_{g} values of the SAD components, e.g., the poorly water soluble active agent and the dispersion polymer. However, T_{g} may vary from that prediction, depending upon the interactions between the components of the SAD, e.g., as calculated by the equations of Couchman-Karasz, Gordon-Taylor, or Fox, among others. T_{g} also depends, in part, on the relative humidity (RH) at which the SAD is stored. Generally, as % RH increases, the T_{g} of the SAD decreases. As T_{g} of the SAD decreases, migration leading to phase separation and/or crystallization of the amorphous poorly water soluble active agent in the SAD increases. Thus, it is beneficial for the SAD to have a sufficiently high T_{g} to minimize or prevent migration and/or crystallization of the amorphous poorly water soluble active agent during the desired shelf life or storage period of the SAD. Advantageously, the T_{g} of the SAD is greater than the temperature at which the SAD is stored. For example, if the SAD is stored at a temperature of 40° C., it is beneficial for the T_{g} of the SAD to be greater than 40° C. under the storage humidity conditions, thereby inhibiting or preventing migration over the desired shelf life or storage period of the SAD. If the T_{g} is lower than the storage temperature, then the SAD may transition to a rubbery or liquid state. For example, the SAD may transition to a rubbery or liquid state over a timeframe that is shorter than the desired shelf life or storage period of the SAD. In some embodiments, the T_{g} of the SAD is at least 10° C. greater than the storage temperature, such as at least 25° C. greater, at least 50° C. greater, or even at least 75° C. greater than the storage temperature. A dispersion polymer with a high T_{g}, such as PMMAMA, facilitates formation of a SAD with a high loading of a poorly water soluble active agent loading that retains a high T_{g}, thereby increasing the physical stability of the SAD relative to a SAD comprising a dispersion polymer with a lower T_{g}. with the same loading of the poorly water soluble active agent. As one example, a SAD comprising 60 wt % erlotinib and 40 wt % PMMAMA having a ~1:1 ratio of free carboxyl groups to ester groups has a T_{g} of 71° C. at 75% RH. In contrast, a comparable SAD comprising HPMCAS-HF instead of PMMAMA has a T_{g} of only 28° C. at 75% RH.

In any or all of the above embodiments, the SAD may further comprise at least one excipient. The SAD may, for example, comprise one or more surfactants, drug complexing agents or solubilizers, lubricants, glidants, fillers, or any combination thereof. In some embodiments, the SAD comprises a surfactant. Surfactants include, for example, sulfonated hydrocarbons and their salts, including fatty acid and alkyl sulfonates, such as sodium 1,4-bis(2-ethylhexyl)sulfosuccinate, also known as docusate sodium (CROPOL) and sodium lauryl sulfate (SLS); poloxamers, also referred to as polyoxyethylene-polyoxypropylene block copolymers (PLURONICs, LUTROLs); polyoxyethylene alkyl ethers (CREMOPHOR A, BRIJ, available from ICI Americas Inc., Wilmington, Del.); polyoxyethylene sorbitan fatty acid esters (polysorbates, TWEEN available from ICI); short-chain glyceryl mono-alkylates (HODAG, IMWITTOR, MYRJ); mono- and di-alkylate esters of polyols, such as glycerol; nonionic surfactants such as polyoxyethylene 20 sorbitan monooleate, (Polysorbate 80, TWEEN 80, available from ICI); polyoxyethylene 20 sorbitan monolaurate (Polysorbate 20, TWEEN 20, available from ICI); polyethylene (40 or 60) hydrogenated castor oil (e.g., CREMOPHOR RH40 and RH60, available from BASF); polyoxyethylene (35) castor oil (CREMOPHOR EL, available from BASF); polyethylene (60) hydrogenated castor oil (Nikkol HCO-60); alpha tocopheryl polyethylene glycol 1000 succinate (Vitamin E TPGS); glyceryl PEG 8 caprylate/caprate (e.g., LABRASOL available from Gattefosse); polyoxyethylene fatty acid esters (e.g., MYRJ, available from ICI), commercial surfactants such as benzethanium chloride (HYAMINE 1622, available from Lonza, Inc., Fairlawn, N.J.); LIPOSORB P-20 polysorbate-40 (available from Lipochem Inc., Patterson N.J.); CAPMUL POE-0 (2-[2-[3,5-bis(2-hydroxyethoxy)oxolan-2-yl]-2-(2-hydroxyethoxy)ethoxy]ethyl (E)-octadec-9-enoate; available from Abitec Corp., Janesville, Wis.), and natural surfactants such as sodium taurocholic acid, 1-palmitoyl-2-oleoyl-sn-glycero-3-phosphocholine, lecithin, and other phospholipids and mono- and diglycerides. Surfactants can advantageously be employed to increase the rate of dissolution by facilitating wetting, thereby increasing the maximum dissolved concentration, and also to inhibit crystallization or precipitation of drug by interacting with the dissolved drug by mechanisms such as complexation, formation of inclusion complexes, formation of micelles or adsorbing to the surface of solid drug. These surfactants may comprise up to 5 wt %, up to 10 wt %, or even up to 15 wt % of the SAD composition. Drug complexing agents or solubilizers include polyethylene glycols, caffeine, xanthene, gentisic acid, and cyclodextrins. Lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate. Glidants include, for example, silicon dioxide, talc, and cornstarch. Fillers or diluents include lactose, mannitol, xylitol, dextrose, sucrose, sorbitol, compressible sugar, microcrystalline cellulose, powdered cellulose, fumed silica, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, and poloxamers such as polyethylene oxide.

In any or all of the above embodiments, the SAD may have a poorly water soluble active agent loading of at least 35 wt %, such as an active agent loading of at least 40 wt %, at least 50 wt %, at least 60 wt %, at least 70 wt %, or at least 75 wt %. In some embodiments, the SAD has a poorly water soluble active agent loading from 35 wt % to 95 wt %, such as 35-90 wt %, 35-85 wt %, 35-75 wt %, 40-75 wt %, 50-75 wt %, or 60-75 wt %. In any or all of the above embodiments, the SAD may include 5-65 wt % matrix material. In some embodiments, the SAD includes 5-60 wt % matrix material, 10-60 wt % matrix material, 10-50 wt % matrix material, 10-40 wt % matrix material, 10-30 wt % matrix material, 10-25 wt % matrix material, or 10-20 wt % matrix material. Where the amounts of active agent and matrix material do not total 100 wt %, the balance of the SAD is comprised of one or more excipients.

In any or all of the above embodiments, particles of the SAD may have an aspect ratio <10, such as an aspect ratio ≤5, ≤4 or ≤3. In some embodiments, at least 95% of the SAD particles have an aspect ratio <10. In certain embodiments, at least 95% or at least 99% of the SAD particles have an aspect ratio AR where 1≤AR<10, 1≤AR≤5, 1≤AR≤4, or 1≤AR≤3. In any or all of the above embodiments, particles of the SAD may have an average diameter, or width at midpoint of the particle length, of 100 μm or less.

### Concentration-Sustaining Polymer

Embodiments of the disclosed SDFs include a SAD as disclosed herein and a concentration-sustaining polymer (CSP). In some embodiments, the CSP is an ionizable cellulosic polymer, a non-ionizable cellulosic polymer, an ionizable non-cellulosic polymer, a non-ionizable non-cellulosic polymer, or a combination thereof. The CSP is not PMMAMA.

Ionizable cellulosic polymers include hydroxypropyl methyl cellulose succinate, cellulose acetate succinate, methyl cellulose acetate succinate, ethyl cellulose acetate succinate, hydroxypropyl cellulose acetate succinate, hydroxypropyl methyl cellulose acetate succinate, hydroxypropyl cellulose acetate phthalate succinate, cellulose propionate succinate, hydroxypropyl cellulose butyrate succinate, hydroxypropyl methyl cellulose phthalate, cellulose acetate phthalate, methyl cellulose acetate phthalate, ethyl cellulose acetate phthalate, hydroxypropyl cellulose acetate phthalate, hydroxypropyl methyl cellulose acetate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, ethyl picolinic acid cellulose acetate, carboxy methyl cellulose, carboxy ethyl cellulose, ethyl carboxy methyl cellulose, and combinations thereof.

Non-ionizable cellulosic polymers include hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose, and combinations thereof.

Ionizable non-cellulosic polymers include carboxylic acid functionalized polymethacrylates, carboxylic acid functionalized polyacrylates, amine-functionalized polyacrylates, amine-functionalized polymethacrylates, proteins, and carboxylic acid functionalized starches, and combinations thereof.

Non-ionizable non-cellulosic polymers include vinyl polymers and copolymers having at least one substituent selected from the group consisting of hydroxyl, alkylacyloxy, and cyclicamido; vinyl copolymers of at least one hydrophilic, hydroxyl-containing repeat unit and at least one hydrophobic, alkyl- or aryl-containing repeat unit; polyvinyl alcohols that have at least a portion of their repeat units in the unhydrolyzed form, polyvinyl alcohol polyvinyl acetate copolymers, polyethylene glycol polypropylene glycol copolymers, polyvinyl pyrrolidone, and polyethylene polyvinyl alcohol copolymers, and combinations thereof.

In some embodiments, the CSP comprises hydroxypropyl methylcellulose acetate succinate (HPMCAS), hydroxypropyl methylcellulose (HPMC), poly(vinylpyrrolidone-co-vinyl acetate) (PVPVA), carboxymethyl ethylcellulose (CMEC), or a combination thereof. In certain embodiments, the CSP comprises HPMCAS or PVPVA. The HPMCAS may be, for example, HPMCAS-HF or Affinisol® 126 HPMCAS polymer (The Dow Chemical Company). HPMCAS-HF has an average particle size of ≤10 μm, such as an average particle size of 5 μm, as measured by laser diffraction. HPMCAS-HF and Affinisol® 126 HPMCAS each have an acetyl content of 10-14 wt %, a succinoyl content of 4-8 wt %, a methoxyl content of 22-26 wt %, and a hydroxypropoxy content of 6-10 wt %. HPCMAS-HF and Affinisol® 126 HPMCAS have an acid content of 0.7 mmol acid/gram and are soluble at pH≥6.5. The PVPVA may be, for example, PVPVA64—a linear random copolymer with a 6:4 ratio of N-vinylpyrrolidone and vinyl acetate. One commercially available example is Kollidon® VA 64 polymer (BASF Corporation). In one embodiment, the active agent is a basic active agent and the CSP comprises HPMCAS. In an independent embodiment, the active agent is a neutral active agent and the CSP comprises PVPVA. Because PVPVA is soluble in gastric media (e.g., at pH 2), PVPVA may retard or prevent crystallization of some active agents in gastric media.

### Solid Dosage Forms

Embodiments of the disclosed solid dosage forms (SDFs) comprise a SAD and a CSP as disclosed herein, wherein the CSP is not dispersed in the SAD. The dispersion polymer in the SAD facilitates rapid disintegration and dissolution of the SDF while the CSP sustains supersaturated drug concentrations in the use environment.

In some embodiments, the SDF further comprises one or more excipients in addition to any excipient(s) that may be present in the SAD. The excipients may include surfactants, pH modifiers, fillers, disintegrants, pigments, binders, lubricants, glidants, flavorants, and so forth for customary purposes and in typical amounts without adversely affecting the properties of the SDF. Surfactants include, for example, sulfonated hydrocarbons and their salts, including fatty acid and alkyl sulfonates, such as sodium 1,4-bis(2-ethylhexyl)sulfosuccinate, also known as docusate sodium (CROPOL) and sodium lauryl sulfate (SLS); poloxamers, also referred to as polyoxyethylene-polyoxypropylene block copolymers (PLURONICs, LUTROLs); polyoxyethylene alkyl ethers (CREMOPHOR A, BRIJ, available from ICI Americas Inc., Wilmington, Del.); polyoxyethylene sorbitan fatty acid esters (polysorbates, TWEEN available from ICI); short-chain glyceryl mono-alkylates (HODAG, IMWITTOR, MYRJ); mono- and di-alkylate esters of polyols, such as glycerol; nonionic surfactants such as polyoxyethylene 20 sorbitan monooleate, (Polysorbate 80, TWEEN 80, available from ICI); polyoxyethylene 20 sorbitan monolaurate (Polysorbate 20, TWEEN 20, available from ICI); polyethylene (40 or 60) hydrogenated castor oil (e.g., CREMOPHOR RH40 and RH60, available from BASF); polyoxyethylene (35) castor oil (CREMOPHOR EL, available from BASF); polyethylene (60) hydrogenated castor oil (Nikkol HCO-60); alpha tocopheryl polyethylene glycol 1000 succinate (Vitamin E TPGS); glyceryl PEG 8 caprylate/caprate (e.g., LABRASOL available from Gattefosse); polyoxyethylene fatty acid esters (e.g., MYRJ, available from ICI), commercial surfactants such as benzethanium chloride (HYAMINE 1622, available from Lonza, Inc., Fairlawn, N.J.); LIPOSORB P-20 polysorbate-40 (available from Lipochem Inc., Patterson N.J.); CAPMUL POE-0 (2-[2-[3,5-bis(2-hydroxyethoxy)oxolan-2-yl]-2-(2-hydroxyethoxy)ethoxy]ethyl (E)-octadec-9-enoate; available from Abitec Corp., Janesville, Wis.), and natural surfactants such as sodium taurocholic acid, 1-palmitoyl-2-oleoyl-sn-glycero-3-phosphocholine, lecithin, and other phospholipids and mono- and diglycerides. Exemplary pH modifiers include acids such as citric acid, acetic acid, ascorbic acid, lactic acid, tartaric acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like; and buffers generally comprising mixtures of acids and the salts of said acids. Fillers or diluents include lactose, mannitol, xylitol, dextrose, sucrose, sorbitol, compressible sugar, microcrystalline cellulose, powdered cellulose, starch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, and poloxamers such as polyethylene oxide. Drug complexing agents or solubilizers include polyethylene glycols, caffeine, xanthene, gentisic acid, and cyclodextrins. Disintegrants include, but are not limited to, sodium starch glycolate, sodium carboxymethyl cellulose, calcium carboxymethyl cellulose, croscarmellose sodium, crospovidone (crosslinked polyvinyl pyrrolidone), methyl cellulose, microcrystalline cellulose, powdered cellulose, starch, pregelatinized starch, and sodium alginate. Exemplary tablet binders include acacia, alginic acid, carbomer, carboxymethyl cellulose sodium, dextrin, ethylcellulose, gelatin, guar gum, hydrogenated vegetable oil, hydroxyethyl cellulose, hydroxypropyl cellulose, hydroxypropyl methyl cellulose, methyl cellulose, liquid glucose, maltodextrin, polymethacrylates, povidone, pregelatinized starch, sodium alginate, starch, sucrose, tragacanth, and zein. Lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate. Glidants include, for example, silicon dioxide, talc, and cornstarch. Other conventional formulation excipients may be employed in the compositions of this invention, including those excipients well-known in the art (e.g., as described in Remington's Pharmaceutical Sciences (16.sup.th ed. 1980).

In some embodiments, the SDF comprises a mixture of particles of the SAD and particles of the CSP, and optionally one or more excipients. The mixture may be formed by any suitable method including, but not limited to, granulation, convective mixing, shear mixing, diffusive mixing, or milling, as described in more detail below. In certain embodiments, the mixture comprises granules of the SAD and CSP. Individual granules may include SAD particles, CSP particles, or a mixture of SAD particles and CSP particles (i.e., an intragranular blend). Mixing conditions are selected so that a molecular dispersion of the poorly water soluble active agent, matrix material, and CSP is not formed. In an independent embodiment, the SAD particles and the CSP particles are present in separate regions of the SDF, e.g., in separate layers.

As discussed above, the poorly water soluble active agent loading in the SAD is at least 35 wt %. In some embodiments, (i) the SDF comprises at least 35 wt % SAD, (ii) the SAD and CSP together comprise at least 50 wt % of the SDF, or (iii) both (i) and (ii). In certain embodiments, the SAD and CSP together are at least 50 wt %, at least 60 wt %, at least 70 wt %, at least 80 wt %, or even at least 90 wt % of the SDF. In some embodiments, the SDF further comprises one or more excipients. For example, the SDF may further comprise excipients in an amount up to 50 wt %, up to 40 wt %, up to 30 wt %, up to 20 wt %, or up to 10 wt %. In some embodiments, the SAD, CSP, and excipients together total 100 wt %.

In some embodiments, the SDF comprises an intragranular (IG) blend comprising SAD particles, CSP particles, and optionally one or more IG excipients (e.g., one or more lubricants, glidants, fillers, or any combination thereof). Individual granules in the IG blend may comprise the SAD, the CSP, one or more IG excipients, or any combination thereof. In certain embodiments, the IG blend includes 0-30 wt % IG excipients, such as 5-30 wt %, 5-25 wt %, 5-20 wt % or 10-20 wt % IG excipients, based on a total mass of the SDF (or, 0-35 wt %, 0-30 wt %, 0-25 wt %, 5-30 wt %, 5-25 wt %, or 10-25 wt % IG excipients based on a total mass of the IG blend). The SDF comprising an IG blend may further include extragranular (EG) excipients, e.g., 0-10 wt %, 1-5 wt %, or 3-5 wt % EG excipients, based on a total mass of the SDF.

In an independent embodiment, the SDF comprises an IG blend comprising SAD particles and one more IG excipients. Individual granules in the IG blend may comprise the SAD, one or more IG excipients, or a combination thereof. In certain embodiments, the IG blend comprises IG excipients in an amount of 0-30 wt % IG, such as 5-30 wt %, 5-25 wt %, 5-20 wt % or 10-20 wt %, based on a total mass of the SDF. In this embodiment, the CSP is extragranular. The SDF may further comprise EG excipients, e.g., in an amount of 0-10 wt %, 1-5 wt %, or 3-5 wt % EG excipients, based on a total mass of the SDF.

In any or all of the above embodiments, the SDF may comprise the SAD in an amount of at least 35 wt %, at least 40 wt %, at least 50 wt %, at least 60 wt %, or at least 70 wt %, such as from 35 wt % to 70 wt % SAD, such as 40-70 wt % SAD, or 40-60 wt % SAD. In any or all of the foregoing embodiments, the SDF may comprise the CSP in an amount of at least 5 wt %, at least 10 wt %, at least 20 wt %, or at least 25 wt %, such as 5-60 wt %, 10-60 wt % CSP, 20-60 wt % CSP, 20-50 wt %, or 20-40 wt % CSP. In any or all of the above embodiments, a ratio of the CSP to the active agent in the SDF may be at least 0.4:1, such as from at least 0.4:1 to as high as a ratio of 5:1, such as from 0.5:1 to 4:1, 0.5:1 to 3:1, or 0.8:1 to 2:1.

In some embodiments, the SDF is a compressed caplet or tablet comprising SAD particles, CSP particles, and optionally one or more excipients. As set forth above, the SAD particles comprise an active agent, a matrix material (i.e., a dispersion polymer), and optionally one or more excipients. In certain embodiments, the SAD particles and CSP particles are granulated together, optionally with one or more excipients, to form a blend, e.g., an intragranular blend. The IG blend is mixed with any desired extragranular excipients and compressed to form the caplet or tablet.

Alternatively, the caplet or tablet may have a layered structure with one or more layers of SAD particles and one or more layers of CSP particles. One or more excipients may be included in the SAD layer(s), the CSP layer(s), or both. In an independent embodiment, the caplet or tablet includes a core comprising SAD particles and, optionally, one or more excipients, and an outer coating comprising the CSP.

In some embodiments, the SDF is a capsule comprising a capsule shell and a fill comprising SAD particles and CSP particles. The fill may further comprise one or more excipients. In certain embodiments, the fill comprises an intragranular blend of the SAD particles, CSP particles, and, optionally, one or more IG excipients. The fill may further comprise one or more extragranular excipients. In such capsules, the capsule shell may comprise any suitable material including, but not limited to, hydroxypropyl methylcellulose, cellulose acetate phthalate, hydroxypropyl methylcellulose acetate succinate, gelatin, starch, casein, chitosan, alginates, gellan gum, carrageenan, xanthan gum, polyvinyl acetate, polyvinyl acetate phthalate, pullulan, and combinations thereof. In an independent embodiment, the SDF is a capsule where the capsule shell comprises the CSP and the fill comprises SAD particles and, optionally, one or more excipients. The fill may, for example, comprise an IG blend of SAD particles and one or more IG excipients, and may further include one or more extragranular excipients.

In any or all of the above embodiments, the oral pharmaceutical composition may further comprise a coating on an outer surface of the SDF, e.g., an enteric coating. Suitable coatings include, but are not limited to, cellulose acetate phthalate, cellulose acetate trimellitate, methylcellulose, ethylcellulose, hydroxyethyl cellulose, gum arabic, carboxymethylcellulose, hydroxypropyl methylcellulose, hydroxypropyl methylcellulose acetate succinate, hydroxypropyl methylcellulose phthalate, hydroxypropyl cellulose, polyvinyl acetate phthalate, shellac, carboxylic acid-functionalized polymethacrylates, carboxylic acid-functionalized polyacrylate, and combinations thereof.

Some embodiments of the disclosed SDFs exhibit greater physical stability than a reference SDF comprising the poorly water soluble active agent in amorphous form and (i) the matrix material (dispersion polymer) alone, (ii) the concentration-sustaining polymer alone, or (iii) a simple mixture of the matrix material and the CSP. By greater physical stability is meant that the amorphous poorly water soluble active agent is less likely to crystallize in the inventive SDF compared to the reference SDF. Greater physical stability is achieved, in part, by increasing the glass transition temperature (T_{g}) of the SAD. As previously mentioned, the T_{g} of the SAD is often approximately equal to a weighted average of the T_{g} values of the SAD components. As the T_{g} of the SAD increases relative to the storage temperature, migration and/or crystallization of the amorphous active agent in the SAD decreases. In certain embodiments, the disclosed SADs comprise a dispersion polymer having a T_{g}≥135° C. at <5% relative humidity. PMMAMA, for example, has a T_{g} up to 190° C. at <5% RH. Other typical dispersion and/or concentration-sustaining polymers often have a much lower T_{g}. For example, the T_{g} of HPMCAS-H is 119° C. at <5% RH. The high T_{g} of PMMAMA facilitates a higher active agent loading in the SAD, compared to a SAD with another dispersion polymer having a lower T_{g}, because the overall T_{g} of the SAD remains sufficiently high to inhibit migration with resulting phase separation and/or crystallization of the active agent over the relevant storage period of the SAD. This benefit is not realized when the amorphous poorly water soluble active agent is merely mixed with PMMAMA.

In some embodiments, PMMAMA is not a sufficiently effective concentration-sustaining polymer. Thus, the SDF further comprises a CSP. Because the CSP is external to the SAD (i.e., the SAD particles do not include the CSP), the CSP does not reduce the T_{g} of the SAD and the physical stability benefits of the SAD are maintained in the SDF. The SAD and CSP may be formulated together into a SDF that comprises a higher active agent loading than a reference SDF that does not include a SAD as disclosed herein. The higher loading allows the SDF to have a smaller overall mass compared to the reference SDF. For example, a reference SAD comprising a poorly water soluble active agent and HPCMAS-H may have an active agent loading of only 35 wt %, whereas an SAD comprising the poorly water soluble active agent and PMMAMA may have an active agent loading of 65 wt %. Thus, if one desired to make a tablet comprising 100 mg of the active agent wherein 50 wt % of the tablet is the SAD, the reference SDF may have a mass of 575 g whereas an SDF as disclosed herein may have a much smaller mass of 300 mg.

The enhanced physical stability and increased poorly water soluble active agent loadings of the disclosed compositions are particularly advantageous when the poorly water soluble active agent is a rapid crystallizer. As the ratio of polymer:active agent is decreased in a reference SDF, the bioavailability may decrease when the SDF enters the intestinal tract due to crystallization of the active agent at the higher pH of the intestinal fluid. Rapid crystallizers frequently dissolve well in gastric media, but then the dissolved concentration rapidly decreases upon entry to the intestinal tract. In contrast, some embodiments of the disclosed oral pharmaceutical compositions provide better in vitro performance compared to a benchmark composition that omits the CSP but is otherwise the same. In certain embodiments, the disclosed oral pharmaceutical composition is expected to provide superior in vivo performance compared to the benchmark composition, such as a greater bioavailability with sustainment of supersaturated dissolved active agent concentrations as discussed in greater detail below.

### III. PREPARATION OF ORAL PHARMACEUTICAL COMPOSITIONS

Embodiments of the disclosed oral pharmaceutical compositions may be prepared by any method that results in a solid dosage form comprising the SAD and the CSP.

In some embodiments, the SAD is formed by spray drying. The spray drying process comprises providing a spray solution comprising the poorly water soluble active agent and the matrix material (e.g., a dispersion polymer such as PMMAMA) in a solvent, introducing the spray solution into an atomizer, atomizing the spray solution into a chamber to form droplets, introducing a drying gas into the chamber to dry the droplets and form a powder comprising particles of the SAD, and collecting the powder from the chamber. In some embodiments, when the matrix material is PMMAMA, the spray solution comprises at least 2 wt %, at least 3 wt %, at least 4 wt %, or at least 5 wt % PMMAMA, such as from 2-9 wt %, 3-9 wt %, 4-9 wt %, or 5-9 wt % PMMAMA. The solvent may be selected from methanol, ethanol, mixtures of acetone and water, mixtures of dichloromethane and ethanol, mixtures of dichloromethane and methanol, mixtures of ethanol and water, mixtures of methanol and water, mixtures of methanol and acetone, mixtures of methanol, acetone and water, mixtures of methyl ethyl ketone and water, or mixtures of tetrahydrofuran and water.

In any or all of the above embodiments, providing the spray solution may comprise dissolving the poorly water soluble active agent and matrix material in the solvent. In some embodiments, the matrix material is dissolved in the solvent and the poorly water soluble active agent is partially dissolved or suspended in the solvent. In any or all of the above embodiments, the process may further comprise dissolving one or more excipients in the spray solution. In certain embodiments, the solvent is selected such that the matrix material, poorly water soluble active agent, and optional excipient(s) are soluble in the solvent. The amount of active agent and/or non-polymer excipients in the spray solution is limited only by practical considerations for spray drying, e.g., solubility of the active/excipients, nozzle clogging, ability to sufficiently dry the spray-dried droplets, etc. In some embodiments, the solids—matrix material, poorly water soluble active agent, and any optional excipients—used to prepare the spray solution comprise from at least 35 wt % active/excipients up to 95 wt % active/excipients, such as from 35 wt % to 85 wt %, from 35 wt % to 80 wt %, or from 35 wt % to 70 wt % active/excipients, with the balance of the solids being the matrix material. In any or all of the above embodiments, the spray solution may have a solids content (matrix material, poorly water soluble active agent, and optional excipients), based on the mass of solids and solvent used to prepare the solution, of from 3 wt % to 40 wt %, such as from 3 wt % to 30 wt %, 3 wt % to 20 wt %, or 3 wt % to 15 wt %. When the matrix material is PMMAMA, the PMMAMA content is from 2-9 wt % as previously described. Advantageously, the concentration of solids is selected so that skinning of the spray solution does not spontaneously occur. In one embodiment, the solids are completely dissolved in the solvent. In an independent embodiment, the solids are substantially dissolved (i.e., at least 90 wt % of the solids is dissolved). In another independent embodiment, all of the matrix material is dissolved and a portion of the active agent and optional excipient(s) is suspended in the spray solution. In some embodiments, the total solids content is from 3-15 wt %, 3-12 wt % or 3-10 wt %.

In any or all of the above embodiments, on a commercial scale, the spray solution may be introduced into the atomizer at a feed rate of at least 3 kg/hr. In some embodiments, the spray solution feed rate is at least 6 kg/hr, at least 10 kg/hr, at least 12 kg/hr, at least 15 kg/hr, or at least 18 kg/hr. The spray solution feed rate may be limited only by practical considerations such as the capacity of the spray-drying apparatus, the nozzle, etc. In some examples, the spray solution feed rate is from 3 kg/hr to 450 kg/hr, such as from 6-450 kg/hr, 10-450 kg/hr, 12-450 kg/hr, 15-450 kg/hr, or 18-405 kg/hr. The drying gas may be introduced into the chamber at a flow rate of at least 72 kg/hr. In some embodiments, the drying gas flow rate is at least 75 kg/hr, at least 100 kg/hr, at least 125 kg/hr, or at least 150 kg/hr. In some examples, the drying gas flow rate is from 72 kg/hr to 2100 kg/hr, such as from 75-2100 kg/hr, 100-2100 kg/hr, 125-2100 kg/hr, or 150-2100 kg/hr. In any or all of the above embodiments, the spray solution feed rate and the drying gas flow rate may be selected to provide a ratio of drying gas flow rate (kg/hr) to spray solution feed rate (kg/hr) of at least 5. In some embodiments, the ratio of drying gas flow rate to spray solution feed rate is from at least 5 to 16, or from at least 8 to 16. A person of ordinary skill in the art of spray drying understands that the foregoing parameters are dependent upon the spray drying apparatus and its capabilities. A smaller spray dryer will typically have lower feed and flow rates. For example, on a smaller, laboratory scale, the spray solution rate may be introduced into the atomizer at a feed rate of at least 1 kg/hr, such as a feed rate of from 1-7 kg/hr with a drying gas flow rate of 30-35 kg/hr. In some instances, the ratio of drying gas flow rate to spray solution gas flow rate may be within a range of from 5-25.

In any or all of the above embodiments, the atomizer may be a pressure nozzle or a two-fluid nozzle. In some embodiments, the pressure nozzle is a pressure-swirl nozzle.

In any or all of the above embodiments, the temperature of the drying gas, when introduced into the chamber, may be <165° C. In some embodiments, the temperature of the drying gas, when introduced into the chamber, is ≤160° C., ≤150° C., ≤125° C., or ≤100° C. In some examples, the temperature of the drying gas, when introduced into the chamber, is from 70-160° C., 80-160° C., 90-160° C., 95-160° C., 95-150° C., or 95-125° C. Suitable drying gases include gases that do not react with the matrix material, the active agent, the solvent, and any other components present in the spray solution (e.g., excipients). Exemplary drying gases include, but are not limited to, nitrogen, argon, and helium. In some embodiments, the drying gas is nitrogen. In one embodiment, the matrix material comprises PMMAMA, the solvent comprises methanol, and the temperature of the drying gas, when introduced into the chamber, is <165° C. In an independent embodiment, the matrix material comprises PMMAMA, the solvent comprises acetone, and the temperature of the drying gas, when introduced into the chamber, is ≤100° C.

In any or all of the above embodiments, the temperature of drying gas at an outlet of the chamber may be <55° C. In some embodiments, the temperature of the drying gas at the outlet is from ambient temperature to <55° C. or from ambient temperature to <50° C. In certain embodiments, the temperature of the drying gas at the outlet of the chamber is at least 50° C. less than the temperature of the drying gas when introduced into the chamber.

In any or all of the above embodiments, the SAD may be mixed with the CSP and optionally one or more excipients to form a mixture. Mixing processes include physical processing, as well as granulation and coating processes. Exemplary mixing methods include granulation, convective mixing, shear mixing, diffusive mixing, or milling. In some embodiments, the mixture is formed by dry granulation, wet granulation, roller compaction/milling or any combination thereof. The mixing conditions are selected to avoid forming a molecular dispersion of the active agent, matrix material, and CSP. In one embodiment, mixing is performed by co-granulating the SAD, the CSP, and optionally one or more excipients. In an independent embodiment, the SAD, CSP, and any excipients are mixed, subjected to roller compaction to provide compressed ribbons, and the compressed ribbons are then milled to provide granules comprising the SAD, CSP, and any excipients. In some embodiments, the mixture comprises (i) an intragranular blend comprising SAD particles, CSP particles, and optionally one or more IG excipients, and (ii) optionally one or more extragranular excipients. The mixture is then formed into the SDF. In one embodiment, the mixture is molded or compressed, as known in the pharmaceutical arts, to provide a tablet or caplet. In an independent embodiment, the mixture is filled into a capsule shell to provide a capsule.

In another independent embodiment, one or more layers of the SAD and one or more layers of the CSP are compressed to form a tablet or caplet. One or more excipients may be included in the SAD layer(s), the CSP layer(s), or both. In yet another independent embodiment, a compressed core comprising the SAD and optionally one or more excipients is formed and coated with a layer comprising the CSP.

In still another independent embodiment, SAD particles, and optionally one or more excipients, are filled into a capsule shell comprising the CSP. The capsule shell may further comprise other components, as known in the pharmaceutical arts, e.g., plasticizers, gelling aids, glidants, lubricants, emulsifiers, and the like.

In any or all of the above embodiments, the oral pharmaceutical composition may comprise the SDF and a coating on an outer surface of the SDF. In some embodiments, the coating is an enteric coating. In certain embodiments, the coating comprises at least one additive selected from lubricants, glidants, pigments, colorants, antifoam agents, antioxidants, waxes, and mixtures thereof. The coating may be applied by any suitable method known in the pharmaceutical arts, including, but not limited to, spray coating (e.g., in a fluidized bed coater or a pan coater), dipping, fluidized bed deposition, and the like.

### IV. USES OF THE ORAL PHARMACEUTICAL COMPOSITIONS

Embodiments of the disclosed oral pharmaceutical compositions are administered to a subject (e.g., a human or animal) for delivery of a poorly water soluble active agent. In some embodiments, the disclosed oral pharmaceutical compositions exhibit a) good physical stability (e.g., with respect to active agent phase separation/crystallization), b) rapid disintegration/dissolution rate, c) sustainment of supersaturated active agent, d) high active agent loading, or any combination thereof. Advantageously, certain embodiments of the oral pharmaceutical compositions provide improved oral bioavailability of poorly water soluble active agents using smaller or fewer dosage units, e.g., a smaller SDF or fewer SDFs may be required to provide the desired dosage of the poorly water soluble active agent.

In any or all of the disclosed embodiments, the SDF, when introduced to a use environment, may provide an initial concentration of the poorly water soluble active agent that exceeds the equilibrium concentration of the poorly water soluble active agent, i.e., a supersaturated concentration, while the CSP retards the rate at which the initial active agent concentration falls to the equilibrium concentration.

Some embodiments of the disclosed SADs, when added to a use environment (e.g., a gastric to intestinal transfer dissolution test) provide a dissolution area under the concentration time curve (AUC) in simulated intestinal fluid, pH 6.5 “SIF”, that is at least 75%, at least 90%, or at least 100% of an AUC of a benchmark composition comprising an SAD comprising the CSP and the poorly water soluble active agent but comprising no PMMAMA, in which the active agent loading in the SAD of the inventive composition is at least 25% greater, at least 40% greater, at least 60% greater, at least 75% greater, or at least 90% greater than the active agent loading in the SAD of the benchmark SDF. The SAD of the disclosed composition is at least as physically stable (e.g. as determined by accelerated stability studies) as the SAD of the benchmark composition. In some embodiments, the disintegration time of the SAD of the disclosed composition, when added to 0.01 N HCl in a USP disintegration apparatus, is ≤10 minutes, such as ≤5 minutes, ≤3 minutes, or ≤2 minutes. The disintegration time may be within a range of 5 seconds to 10 minutes, 5 seconds to 5 minutes, 5 seconds to 3 minutes, or 5 seconds to 2 minutes.

Some embodiments of the disclosed SDFs, when added to a use environment (e.g., a gastric to intestinal transfer dissolution test) provide a dissolution area under the concentration time curve (AUC) in simulated intestinal fluid, pH 6.5 “SIF”, that is at least 75%, at least 90% or at least 100% of an AUC of a benchmark SDF for which the SDF of the disclosed composition and the SDF of the benchmark composition contain the same amount of CSP (e.g. within +5%), but for which the active agent loading in the SDF of the disclosed composition is at least 25% greater, at least 40% greater, at least 60% greater, at least 75% greater, or at least 90% greater than the active agent loading in the SDF of the benchmark composition. The benchmark SDF comprises (i) an SAD comprising the active agent and the CSP, but no PMMAMA and (ii) additional excipients, but no CSP, external to the SAD. The embodiment of the disclosed SDF comprises (i) an SAD comprising the active agent and PMMAMA, but no CSP and (ii) CSP and additional excipients external to the SAD. The SAD of the disclosed composition is at least as physically stable (e.g. as determined by accelerated stability studies) as the SAD of the benchmark composition. In some embodiments, the disintegration time of the SDF of the disclosed composition, when added to 0.01 N HCl in a USP disintegration apparatus, is ≤10 minutes, such as ≤5 minutes, ≤3 minutes, or ≤2 minutes. The disintegration time may be within a range of 5 seconds to 10 minutes, 5 seconds to 5 minutes, 5 seconds to 3 minutes, or 5 seconds to 2 minutes. In certain examples, the disintegration time of the SDF of the disclosed composition may be the same as or less than the disintegration time of the SDF of the benchmark composition.

Some embodiments of the disclosed SDFs, when added to a use environment (e.g., a gastric to intestinal transfer dissolution test as described in the Methods section below) provide a dissolution area under the concentration time curve (AUC) in simulated intestinal fluid, pH 6.5 “SIF”, that is at least 75%, at least 90% or at least 100% of an AUC of a benchmark SDF for which the SDF of the disclosed composition contains a ratio of CSP:drug that is less than that of the SDF of the benchmark composition (e.g., the CSP:drug ratio of the disclosed SDF at least 40%, at least 50%, at least 70%, or at least 90% less than the CSP:drug ratio of the benchmark SDF), but for which the active agent loading in the SDF of the disclosed composition is at least 25% greater, at least 40% greater, at least 60% greater, at least 75% greater, or at least 90% greater than the active agent loading in the SDF of the benchmark composition. The benchmark SDF comprises (i) an SAD comprising the active agent and the CSP, but no PMMAMA and (ii) additional excipients, but no CSP, external to the SAD. The embodiment of the disclosed SDF comprises (i) an SAD comprising the active agent and PMMAMA, but no CSP and (ii) CSP and additional excipients external to the SAD. The SAD of the disclosed composition is at least as physically stable (e.g. as determined by accelerated stability studies) as the SAD of the benchmark composition. In some embodiments, the disintegration time of the SDF of the disclosed composition, when added to 0.01 N HCl in a USP disintegration apparatus, is ≤10 minutes, such as ≤5 minutes, ≤3 minutes, or ≤2 minutes. The disintegration time may be within a range of 5 seconds to 10 minutes, 5 seconds to 5 minutes, 5 seconds to 3 minutes, or 5 seconds to 2 minutes. In certain examples, the disintegration time of the SDF of the disclosed composition may be the same as or less than the disintegration time of the SDF of the benchmark composition.

In any or all of the above embodiments of the disclosed SDFs, when the disclosed SDF is added to a use environment (e.g., a gastric to intestinal transfer dissolution test) it may provide a dissolution area under the concentration time curve (AUC) in simulated intestinal fluid, pH 6.5 “SIF”, that is at least 125%, at least 150%, at least 200%, at least 400%, or at least 600% that of an AUC of an SDF of a control composition comprising the same SAD (e.g. the active agent and PMMAMA, but no CSP) but no CSP in the SDF, wherein a wt % of the SAD in the disclosed composition is equal to a wt % of the SAD in the SDF of the control composition, and the active agent loading in the SDF of the disclosed composition is equal to the active agent loading in the SDF of the control composition.

In any or all of the above embodiments of the disclosed SDFs, when added to a use environment (e.g., a gastric to intestinal transfer dissolution test) may provide a dissolution area under the concentration time curve (AUC) in simulated intestinal fluid, pH 6.5 “SIF”, that is at least 125%, at least 150%, at least 200%, at least 300%, or at least 400% that of an AUC of an SDF of a control composition comprising an SAD comprising the poorly water soluble active agent and the CSP but comprising no PMMAMA, wherein the wt % of active agent in the SAD in the disclosed composition is equal to the wt % of SAD in the control composition, the wt % SAD in the SDF of the disclosed composition is equal to the wt % of SAD in the SDF of the control composition, the wt % of CSP in the SDF of the disclosed composition is equal to the wt % of the CSP in the SDF of the control composition and the active agent loading in the SDF of the disclosed composition is equal to the active agent loading in the SDF of the control composition. The SAD of the disclosed composition is more physically stable (e.g. as determined by accelerated stability studies) than the SAD of the control composition. In some embodiments, the disintegration time of the SDF of the disclosed composition, when added to 0.01 N HCl in a USP disintegration apparatus, is ≤10 minutes, such as ≤5 minutes, ≤3 minutes, or ≤2 minutes. The disintegration time may be within a range of 5 seconds to 10 minutes, 5 seconds to 5 minutes, 5 seconds to 3 minutes, or 5 seconds to 2 minutes. In certain examples, the disintegration time of the SDF of the disclosed composition may be the same as or less than the disintegration time of the SDF of the benchmark composition.

### V. REPRESENTATIVE EMBODIMENTS

Representative, non-limiting embodiments of the disclosed oral pharmaceutical compositions are shown in the following numbered paragraphs.
1. An oral pharmaceutical composition comprising a solid dosage form (SDF), the SDF comprising: a solid amorphous dispersion (SAD) comprising a poorly water soluble active agent and a matrix material comprising poly[(methyl methacrylate)-co-(methacrylic acid)](PMMAMA) having a glass transition temperature T_{g}≥135° C. at <5% relative humidity as measured by differential scanning calorimetry; and a concentration-sustaining polymer (CSP), wherein the CSP is not PMMAMA, the CSP is not dispersed in the SAD, and the SAD is at least 35 wt % of the SDF.
2. The oral pharmaceutical composition of paragraph 1, wherein the CSP comprises hydroxypropyl methylcellulose acetate succinate (HPMCAS), hydroxypropyl methylcellulose (HPMC), poly(vinylpyrrolidone-co-vinyl acetate) (PVPVA), carboxymethyl ethylcellulose (CMEC), or a combination thereof.
3. The oral pharmaceutical composition of paragraph 1 or paragraph 2, wherein the poorly water soluble active agent has a melting temperature Tₘ to glass transition temperature T_{g} ratio ≥1.3, ≥1.35 or ≥1.4, and a Log P<10.
4. The oral pharmaceutical composition of any one of paragraphs 1-3, wherein the SAD has an active agent loading of at least 35 wt %, at least 40 wt %, at least 50 wt %, at least 60 wt %, at least 70 wt %, or even at least 75 wt %.
5. The oral pharmaceutical composition of paragraph 4, wherein the SAD is at least 40 wt % of the SDF, at least 50 wt % of the SDF, at least 60 wt %, or even at least 70 wt % of the SDF.
6. The oral pharmaceutical composition of any one of paragraphs 1-5, wherein the CSP is at least 5 wt % of the SDF, at least 10 wt % of the SDF, at least 20 wt % of the SDF, or even at least 25 wt % of the SDF.
7. The oral pharmaceutical composition of any one of paragraphs 1-6, wherein the SAD and the CSP together are at least 50 wt % of the SDF, at least 60 wt % of the SDF, at least 70 wt % of the SDF, at least 80 wt % of the SDF, or even at least 90 wt % of the SDF.
8. The oral pharmaceutical composition of any one of paragraphs 1-7, wherein a ratio of the CSP to the active agent is from 0.4:1 to 5:1, 0.5:1 to 3:1, or even 0.8:1 to 2:1.
9. The oral pharmaceutical composition of any one of paragraphs 1-8, wherein the PMMAMA has a free carboxyl group to ester group ratio of from 1:0.8 to 1:2.2.
10. The oral pharmaceutical composition of any one of paragraphs 1-9 wherein at least 95% of particles of the SAD have an aspect ratio <10.
11. The oral pharmaceutical composition of any one of paragraphs 1-10, wherein the SAD further comprises at least one excipient.
12. The oral pharmaceutical composition of any one of paragraphs 1-11, wherein the SDF comprises: a granular blend comprising particles of the SAD and particles of the CSP; or an intragranular blend wherein individual granules comprise SAD particles and CSP particles.
13. The oral pharmaceutical composition of paragraph 12, wherein at least some of the individual granules of the intragranular blend comprise SAD particles, CSP particles, and one or more intragranular excipients.
14. The oral pharmaceutical composition of paragraph 12 or paragraph 13, wherein the SDF further comprises one or more extragranular excipients.
15. The oral pharmaceutical composition of any one of paragraphs 1-14, wherein the SDF is a compressed tablet or caplet, wherein the SAD and CSP are blended and compressed to form the tablet or caplet.
16. The oral pharmaceutical composition of any one of paragraphs 1-14, wherein the SDF is a compressed tablet or caplet comprising compressed SAD particles and an outer coating comprising the CSP.
17. The oral pharmaceutical composition of any one of paragraphs 1-14, wherein the SDF is a capsule comprising a capsule shell and a fill comprising the SAD and the CSP.
18. The oral pharmaceutical composition of any one of paragraphs 1-14, wherein the SDF is a capsule comprising a capsule shell comprising the CSP and a fill comprising the SAD.

### VI. EXAMPLES

### General Methods

Dissolution Performance: Tablets and suspensions were evaluated for dissolution performance in a gastric to intestinal transfer dissolution test using a USP 2 dissolution apparatus (Vankel VK 7000, Agilent, Santa Clara, CA) with fiber optic UV probe detection (Rainbow™, Pion, Billerca, MA). Prior to the experiment, unique calibration curves were built for each UV probe (2 mm path length) by delivering aliquots of a known amount of stock API solution (10-15 mg/mL erlotinib in methanol or 10-15 mg/mL posaconazole in 95/5 THF/H₂O) to 50-100 mL of simulated gastric fluid (SGF), consisting of 0.01N HCl, pH 2.00, or simulated intestinal fluid (SIF), consisting of 67 mM potassium phosphate at pH 6.50+0.5 wt % FaSSIF/FeSSIF/FaSSGF powder (Biorelevant.com, London, United Kingdom) held at 37±2° C. HPMC E3 was added to the SIF solution when making standards to sustain the supersaturated erlotinib solutions. To begin dosing, one tablet was added to 200 mL of SGF contained within a 500 ml USP 2 dissolution vessel to achieve a nominal dose concentration of 500 μg/mL erlotinib. Samples were stirred at 75 rpm and held at 37° C. by circulating water through a heating block mounted to the USP 2 dissolution apparatus. Dissolution performance in SGF was monitored for 30 minutes via UV probes using a wavelength range of 386-396 nm (2^{nd} derivative spectra) within a calibration range of 0-550 μg/ml. After 30 minutes, 200 ml of 134 mM phosphate at pH 6.55+1.0 wt % FaSSIF/FeSSIF/FaSSGF powder was added to the dissolution vessel to achieve a final dose concentration of 250 μg/mL in 400 ml of SIF. Dissolution performance in SIF was monitored over the course of 90 minutes using a wavelength range of 366-376 nm (2^{nd} derivative spectra) within a calibration range of 0-290 μg/mL for erlotinib or a wavelength range of 266-272 nm (2^{nd} derivative spectra) within a calibration range of 0-160 μg/mL for posaconazole. Area under the curve was calculated using the trapezoidal method using the dissolution profiles in SIF.

Disintegration Performance: Tablets were evaluated for disintegration performance in a USP (See general chapter <701>) disintegration apparatus (ZT-71 disintegration tester, Erweka, Heusenstamm, Germany), consisting of a basket-rack assembly contained within a 1000-ml low-form beaker. Tablets were placed one each inside one of the six tubes within the basket-rack assembly. A disk was then added on top of each tablet. The beaker was filled with 750 ml of 0.01N hydrochloric acid as the immersion fluid, which was maintained at 37±2° C. To start the test, the basket-rack assembly was automatically raised and lowered within the immersion fluid at a constant frequency through a fixed distance as specified in USP <701>. The time at which the disk touched the wire mesh at the bottom of the tube (e.g. the tablet had sufficiently broken into fragments and fallen through the mesh) as automatically detected by the apparatus was noted as the disintegration time.

Accelerated Stability Studies: The samples were stored under elevated temperature and humidity conditions to increase the rate of physical changes occurring in the materials in order to simulate a longer storage interval in a typical storage environment. Approximately 100 mg of each material was transferred to a 4 mL glass vial. Each vial was then covered with perforated aluminum foil and transferred to a temperature/humidity controlled oven (Environmental Specialties Inc., Model ES2000) at 50° C. and 75% relative humidity and allowed to stand undisturbed for 7, 14 and 28 days. Other conditions tested included 40° C./75% RH and 50° C./45% RH. Samples were then removed from the oven and transferred to a vacuum dessicator for up to 18 hours to remove adsorbed water from the samples. The samples were then removed from the vacuum dessicator and tightly capped and stored at 5° C. Analysis of crystallinity using SEM and pXRD and analysis of Tg using DSC were done before and after such storage in order to evaluate stability of the dispersions.

Differential Scanning Calorimetry (DSC): Samples were analyzed to confirm that they were homogeneous as evidenced by a single glass transition temperature (Tg) using a TA Instruments Q2000 modulated differential scanning calorimeter (TA Instruments-Waters L.L.C, New Castle, DE). Samples were prepared as loose powder, loaded into a Tzero pan (TA Instruments) and equilibrated at <5% RH for up to 18 hours. Samples were then crimped with hermetic lids and was run in modulated mode at a scan rate of 2.5° C./min, modulation of 1.5° C./min, and a scan range −20 to 200° C.

Scanning Electron Microscopy (SEM): The materials were assessed for the presence of crystals and changes in particle shape and morphology, before and after exposure to increased temperature and humidity, using SEM analysis as described below. Approximately 0.5 mg of sample was mounted to an aluminum stub with 2-sided carbon tape. The sample was sputter-coated (Hummer Sputtering System, Model 6.2, Anatech Ltd.) with an Au/Pd stage for 10 minutes at 15 mV, and studied by SEM. Samples before aging generally appear as spheres or collapsed spheres with smooth and rounded faces and surfaces. Changes in particle appearance indicating physical instability include: fusing together of individual particles, changes in surface texture, changes in general particle shape, and appearance of straight edges in the particle (indicating possible crystallinity).

Powder X-Ray Diffraction (PXRD): Samples were analyzed using powder X-ray diffraction to confirm they were amorphous, as evidenced by the lack of sharp Bragg diffraction peaks in the x-ray pattern, using a Rigaku MiniFlex600 X-Ray Diffractometer (Rigaku, The Woodlands, TX) equipped with a Cu-Kα source. The scan rate was set to 2.5°/min with a 0.02° step size from 3° to 400 2Θ.

### Example 1

### High Loaded Dosage Forms (HLDF) with Erlotinib

Erlotinib is a rapid crystallizer with poor physical stability when included as the amorphous form in a SDF with a high drug loading. Common dosages of erlotinib are 150 mg/day (non-small cell lung cancer) and 100 mg/day (pancreatic cancer). Erlotinib has the following measured properties: Log P 2.8, pKa (base) 5.3, crystalline solubility in 0.5% simulated intestinal fluid (SIF) 3 g/mL, crystalline solubility in gastric buffer (GB) 182 g/mL, amorphous solubility in 0.5% SIF~380 g/mL, Tₘ 157° C., T_{g} 39° C., Tₘ/T_{g} (K/K) 1.4.

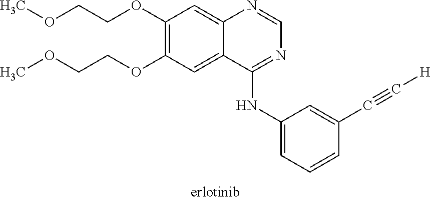 [SMILES: C#Cc1cccc(Nc2ncnc3cc(OCCOC)c(OCCOC)cc23)c1]

Spray solutions were prepared by dissolving erlotinib and a dispersion polymer (PMMAMA or hydroxypropyl methylcellulose acetate succinate H grade) in methanol at the desired ratio of erlotinib to polymer at a solids loading of 3%. Solutions were spray dried with an outlet temperature of 45-50° C. and an inlet temperature of 150-160° C. on a customized spray dryer (suitable for batch sizes from 0.5-200 grams) capable of drying gas flow rates of up to 35 kg/hr using a pressure swirl Schlick 2.0 spray nozzle (Düsen-Schlick GmbH, Untersiemau, Germany). After the spray drying process, spray dried dispersions were placed in a Gruenberg Benchtop Lab Dryer (Thermal Product Solutions, New Columbia, PA) for >18 hr at 35-40° C. to remove residual solvent.

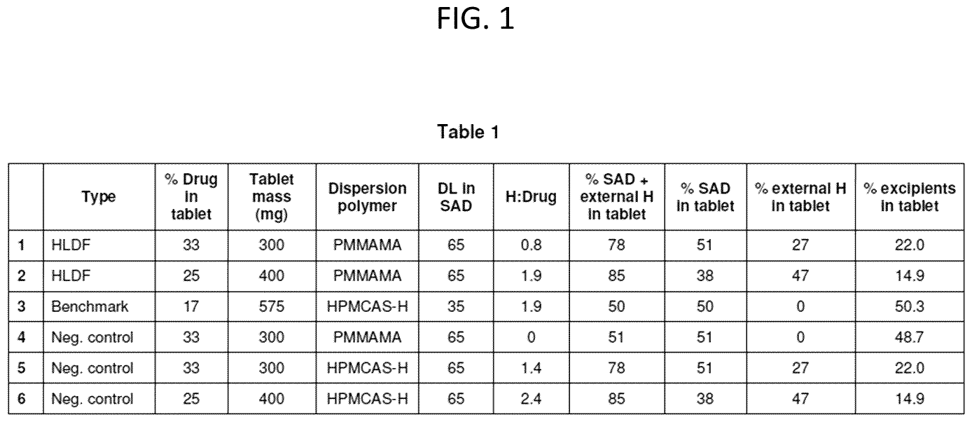

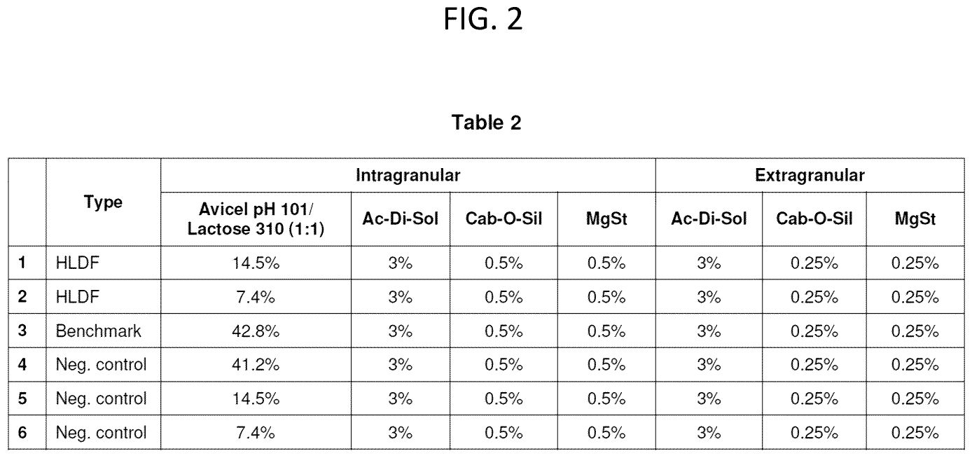

Tablet compositions 1-6 including 100 mg erlotinib were prepared as shown in Table 1 (FIG. 1), where SAD=spray-dried solid amorphous dispersion, DL=drug loading, H=HPMCAS-HF, and “external H” refers to HPMCAS-HF that is external to the SAD. The tablets included excipients as shown in Table 2 (FIG. 2). The excipients were a 1:1 blend of Avicel® PH-101 microcrystalline cellulose (a filler, available from DuPont Nutrition & Health) and Lactose 310 (a filler, available from UPI Chem., Somerset, NJ)), Ac-Di-Sol (croscarmellose sodium, a disintegrant, available from DuPont Nutrition & Health) Cab-O-Sil® fumed silica (a filler, available from Cabot Corporation, Alpharetta, GA), and magnesium stearate (MgSt; a lubricant).

The tablet compositions were made by preparing an intragranular (IG) blend of (i) a spray-dried SAD comprising erlotinib and a dispersion polymer (PMMAMA (i.e., Eudragit® L100 polymer, hereinafter “PMMAMA-1”; or HPMCAS-H) as indicated in Table 1, (ii) HPMCAS-HF (except for compositions 3 and 4), and (iii) IG excipients as indicated in Table 2. The IG blend was then blended with extragranular (EG) excipients as shown in Table 2 and compressed to form a tablet.

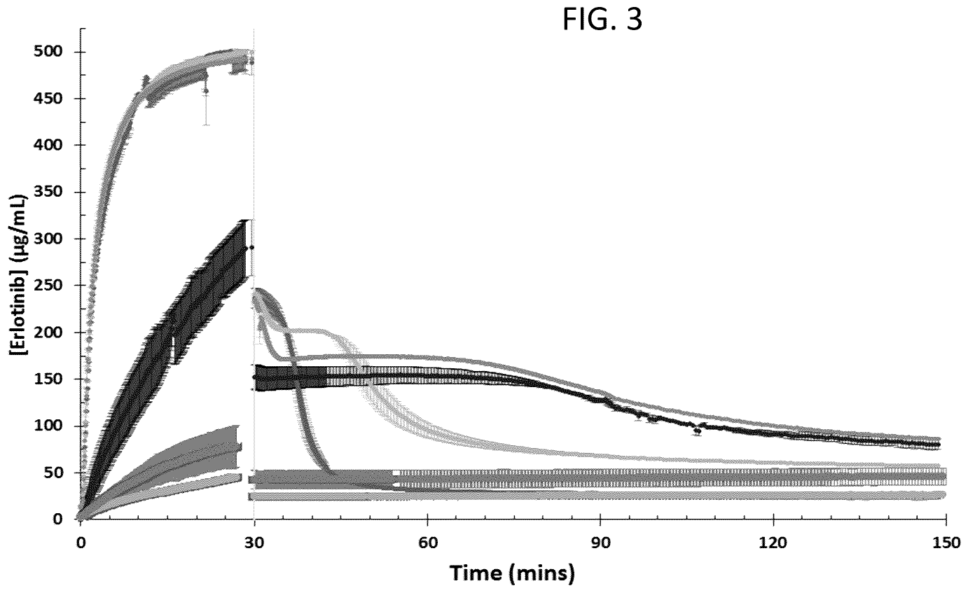

The tablet compositions were evaluated for dissolution performance and disintegration time (in 0.01 N HCl) as described in the Methods. The results are shown in Table 3 and FIG. 3. The maximum possible dissolved concentration during the gastric portion of the dissolution test was 500 g/mL based on the mass of active agent and the volume of 0.01 N HCl. An additional negative control (not shown in Table 3) was made by increasing the percentage of 35:65 erlotinib:HPMCAS-H SAD in the benchmark tablet to 70% to provide a 400-mg tablet comprising 25 wt % erlotinib. This tablet composition had a very long disintegration time (>1 h) and poor dissolution performance (not shown).

TABLE 3

| | Tablet | AUC from 30-90 min. | Disintegration time |
|---|---|---|---|
| | type | (μg*min/mL) | (h:min:s) |
| 1 | HLDF | 9071-9721 | 0:00:40 |
| 2 | HLDF | 13542-13649 | 0:00:47 |
| 3 | Benchmark | 11723-12586 | 0:00:56 |
| 4 | Neg. ctrl. | 4165-4525 | 0:00:18 |
| 5 | Neg. ctrl. | 2120-2436 | >1:00:00 |
| 6 | Neg. ctrl. | 3095-4823 | >1:00:00 |

### Example 2

### Manufacturing Study for HLDFs with Erlotinib

Tablets according to compositions 1 and 2 (Tables 1 and 2; FIGS. 1 and 2) were formulated by two different approaches. The first approach is described in Example 1. Briefly, the SAD, HPMCAS-HF and IG excipients were combined to form an IG blend. The IG blend was then mixed with EG excipients and compressed to form a tablet. In the second approach, the SAD and IG excipients were combined to form an IG blend. The IG blend was then mixed with EG excipients and HPMCAS-HMP (medium particle size grade, Shin-Etsu AQOAT Grade: AS-HMP), and compressed to form a tablet. Thus, the two approaches differed in grade of HPMCAS—fine or medium particle size—and the location of the HPMCAS—in the IG blend (internal) or external to the IG blend. The formulations are summarized in Table 4.

TABLE 4

| | 7 | 8 | 9 | 10 |
|---|---|---|---|---|
| Processing strategy* | Internal | Internal | External | External |
| % Drug in tablet | 33 | 25 | 33 | 25 |
| Tablet mass (mg) | 300 | 400 | 300 | 400 |
| Dispersion polymer | PMMAMA-1 | PMMAMA-1 | PMMAMA-1 | PMMAMA-1 |
| Drug loading in SAD | 65 | 65 | 65 | 65 |

*Internal (HPMCAS in IG blend) or External (HPMCAS extragranular)

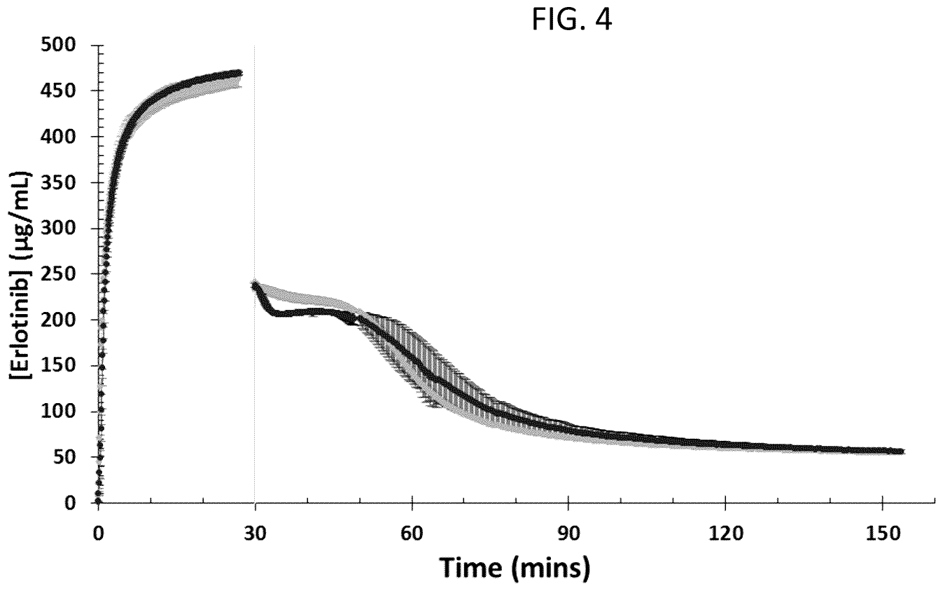

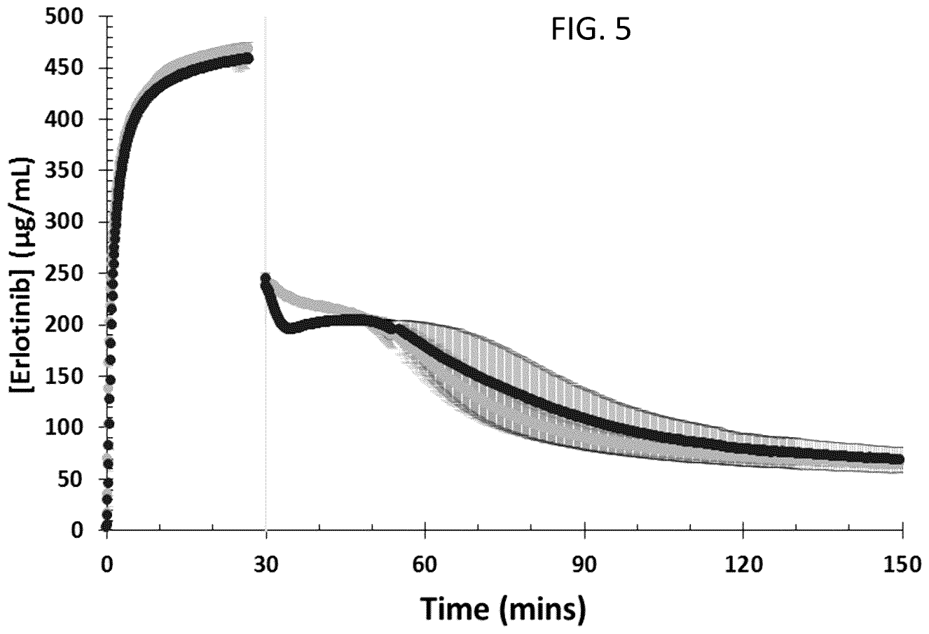

The dissolution performance of the tablets was evaluated as described in Methods. The results are shown in FIG. 4 (300 mg tablets) and FIG. 5 (400 mg tablets). The results show that similar in vitro performance is obtained, and the CSP may be included in the IG blend or external to the IG blend with similar in vitro effect.

### Example 3

### Physical Stability of SDDs with Erlotinib and PMMAMA-1 or HPMCAS-H

Spray-dried dispersions including different drug loadings (erlotinib) and a dispersion polymer—HPMCAS-H or PMMAMA-1—were prepared and subjected to accelerated physical stability studies as described in Methods. Drug loadings ranged from 25-75 wt % in PMMAMA-1 and 25-60 wt % in HPMCAS-H. In the stability studies, the SADs were placed in open containers inside a chamber set to a specified temperature and relative humidity. Samples of the SDDs were removed from the chambers at 0, 1, 2, and 4 weeks and evaluated via:
Differential scanning calorimetry (DSC) to measure the glass transition temperature (T_{g}) and potential crystallization or melting events;
Powder x-ray diffraction (PXRD) to measure the presence of crystallinity (down to ~3% of sample mass); and
Scanning electron microscopy (SEM) to detect visual changes in morphology, fusing of SADs, and/or the presence of crystals.

A summary of the results is presented in Table 5, where DL=drug loading and RH=relative humidity. Examples 16-19 are benchmark compositions that do not include PMMAMA.

TABLE 5

| | Dispersion | % DL | | |
|---|---|---|---|---|
| | polymer | in SAD | Conditions | Results |
| 11 | PMMAMA-1 | 25 | 40° C., 75% RH | Stable (no change) |
| 12 | PMMAMA-1 | 50 | 40° C., 75% RH | Stable (no change) |
| 13 | PMMAMA-1 | 60 | 40° C., 75% RH | Stable (no change) |
| 14 | PMMAMA-1 | 65 | 40° C., 75% RH | Stable (no change) |
| | | | and | |
| | | | 50° C., 75% RH | |
| 15 | PMMAMA-1 | 75 | 40° C., 75% RH | Less stable |
| | | | | increased ordering |
| | | | | after 1 week |
| 16 | HPMCAS-H | 25 | 40° C., 75% RH | Stable (no change) |
| 17 | HPMCAS-H | 35 | 40° C., 75% RH | Stable (no change) |
| | | | and | |
| | | | 50° C., 75% RH | |
| 18 | HPMCAS-H | 50 | 40° C., 75% RH | Unstable - crystals |
| | | | | after 1 week |
| 19 | HPMCAS-H | 60 | 40° C., 75% RH | Unstable - crystals |
| | | | | after 1 week |

The results show that spray-dried SADs comprising PMMAMA-1 remained stable (i.e., the drug remained amorphous) for at least 4 weeks at drug loadings up to at least 65 wt %. Benchmark SADs comprising HPMCAS-H remained stable for at least 4 weeks at drug loadings up to 35 wt %; however, at drug loadings of 50-60 wt %, the benchmark SADs showed instability after just 1 week under the study conditions. Thus, PMMAMA provided superior stability at higher drug loadings than the benchmark dispersion polymer HPMCAS-H.

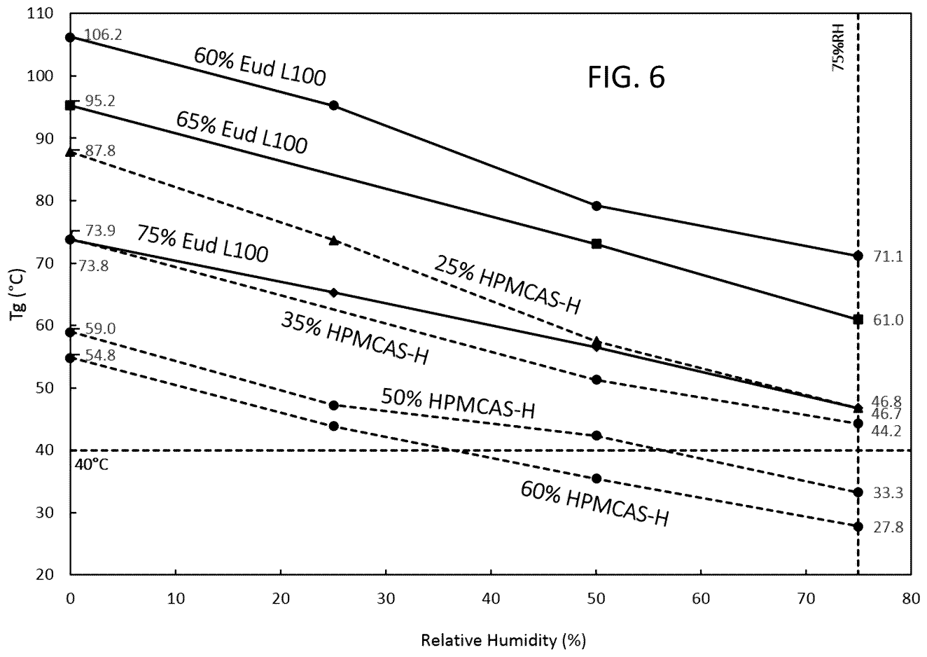

FIG. 6 is a graph showing the glass transition temperature T_{g} of the SADs as a function of relative humidity (RH); EUD L100=Eudragit® L100 PMMAMA polymer. The T_{g} of Eudragit® L100 PMMAMA is 191° C.; the T_{g} of HPMCAS-H is 121° C. The results show that, at a given drug loading and % RH, PMMAMA-based SADs have higher T_{g} values than HPMCAS-H-based SADs. The results also show that HPMCAS-H-based SADs with 50 wt % (composition 18) and 60 wt % (composition 19) drug loadings have T_{g} values less than the accelerated stability storage temperature (40° C.) when the RH is 75%, which explains the poor stability of these SADs. In contrast, the Eudragit® L100 PMMAMA-based SADs (compositions 12, 13, 15) all have T_{g} values greater than the accelerated stability storage temperature (40° C.) at 75% RH, providing the PMMAMA-based SADs with greater storage stability.

### Example 4

### HLDFs with Erlotinib and PMMAMA-1 or PMMAMA-2

HLDFs were prepared with erlotinib in PMMAMA-1 or PMMAMA-2 (Eudragit® S100 polymer) In each HLDF, the drug loading in the spray-dried SAD was 65 wt %, and the CSP was HMCAS-HF incorporated into the intragranular blend.

TABLE 6

| | Dry T_{g} | Acid content |
|---|---|---|
| Polymer | (° C.) | (mol/100 g) |
| PMMAMA-1 | 191 | 0.54 |
| PMMAMA-2 | 172 | 0.35 |
| HPMCAS-L, -M, -H | 121 | 0.15, 0.11, 0.06 |

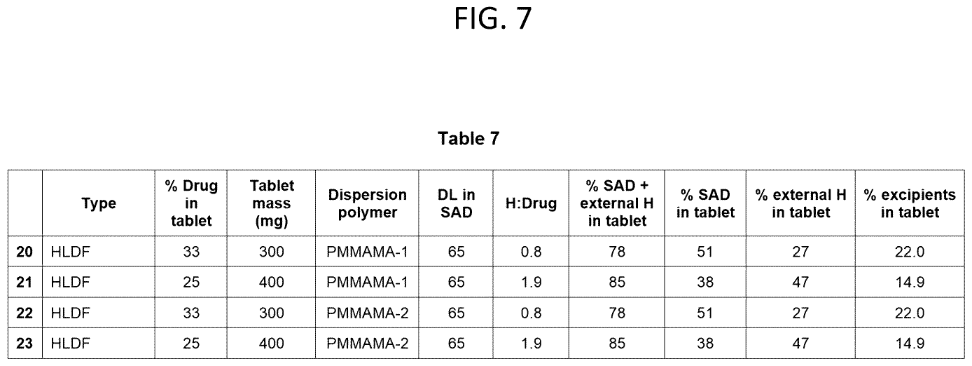

Tablets including 33 wt drug and 25 wt % drug were prepared as shown in Table 7 (FIG. 7), where H=intragranular HPMCAS-HF. The excipients are those disclosed in Table 2 (FIG. 2) for compositions 1 (33 wt % drug) and 2 (25 wt % drug).

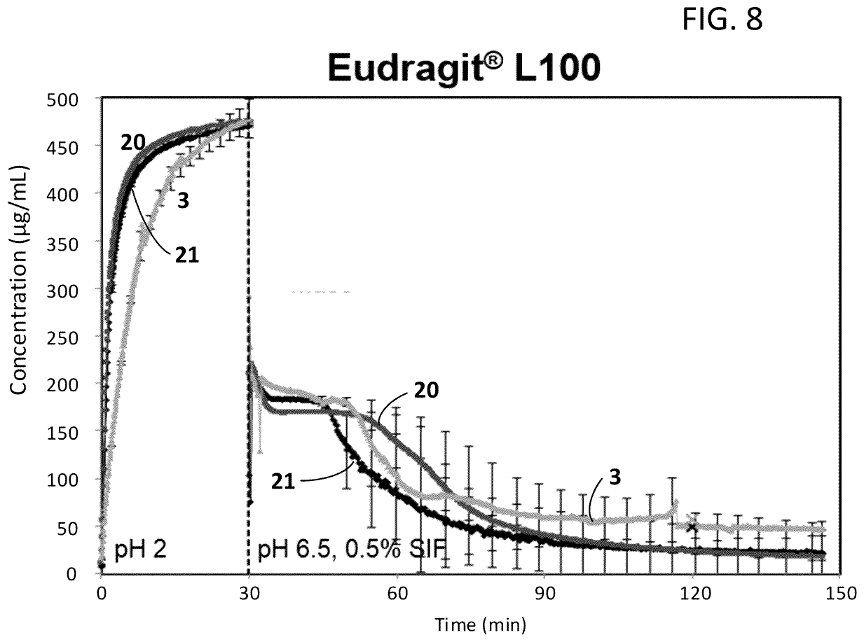

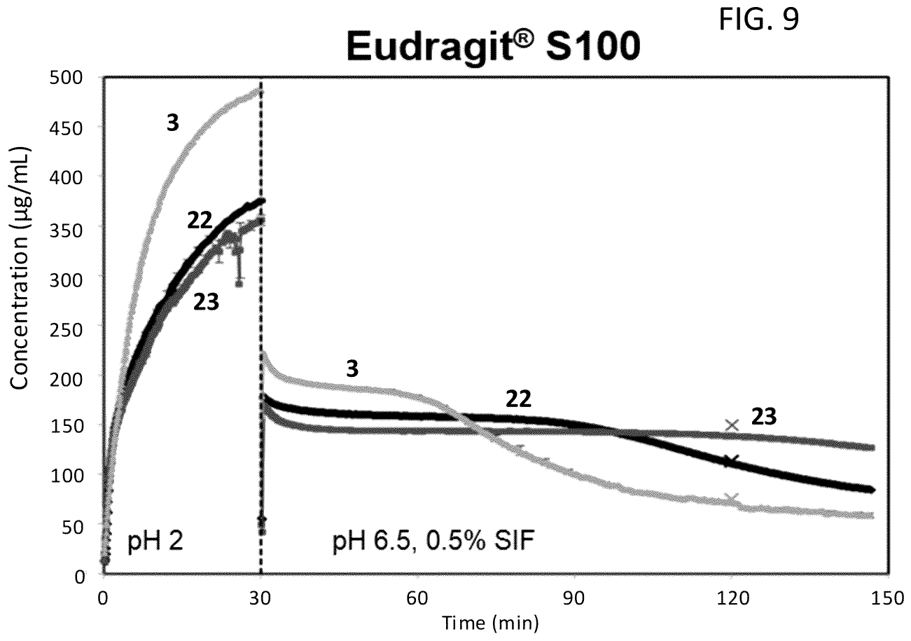

Disintegration and dissolution tests were performed as described in Methods. The disintegration results are shown in Table 8. Composition 3 is a benchmark 575 mg tablet including 17 wt % active (see Table 1). The in vitro dissolution results are shown in FIGS. 8 and 9: PMMAMA-1 (Eudragit® L100) (FIG. 8), PMMAMA-2 (Eudragit® S100) (FIG. 9).

TABLE 8

| | Dispersion | Drug mass | % Drug | % Drug | Disintegration | AUC |
|---|---|---|---|---|---|---|
| | Polymer | (mg) | in SAD | in tablet | time (h:min:s) | μg/mL · min |
| 20 | PMMAMA-1 | 100 | 65 | 33 | 0:00:40 | 10489-12221 |
| 21 | PMMAMA-1 | 100 | 65 | 25 | 0:00:47 | 11108-14808 |
| 22 | PMMAMA-2 | 100 | 65 | 33 | 0:00:34 | 11771-12104 |
| 23 | PMMAMA-2 | 100 | 65 | 25 | 0:00:42 | 11343-11157 |
| 3 | HPMCAS-H | 100 | 35 | 17 | 0:00:56 | 11385-12179 |

The results show that the HLDFs with PMMAMA-1 and PMMAMA-2 had similar disintegration times and similar performance to the benchmark composition in the intestinal portion of the test (post 30 minutes).

Accelerated stability tests were performed as described in Methods at 50° C. and 7500 RH. A reference SAD comprising 35 wt %0 erlotinib in HPMCAS-H was used as a comparison. The results are summarized in Table 9. From a physical stability standpoint, Eudragit® S100 polymer (PMMAMA-2) was inferior to Eudragit® L100 polymer (PMMAMA-1) at an erlotinib loading of 65 wt % in the SAD.

TABLE 9

| | Dispersion | DL in | | | |
|---|---|---|---|---|---|
| | Polymer | SAD | 1 week | 2 weeks | 4 weeks |
| 20 | PMMAMA-1 | 65 | stable | stable | stable |
| 51 | PMMAMA-1 | 65 | stable | stable | stable |
| 22 | PMMAMA-2 | 65 | stable | Less stable | Less stable |
| | | | | increased | increased |
| | | | | ordering | ordering |
| 23 | PMMAMA-2 | 65 | stable | Less stable | Less stable |
| | | | | increased | increased |
| | | | | ordering | ordering |
| 3 | HPMCAS-H | 35 | stable | stable | fusing |

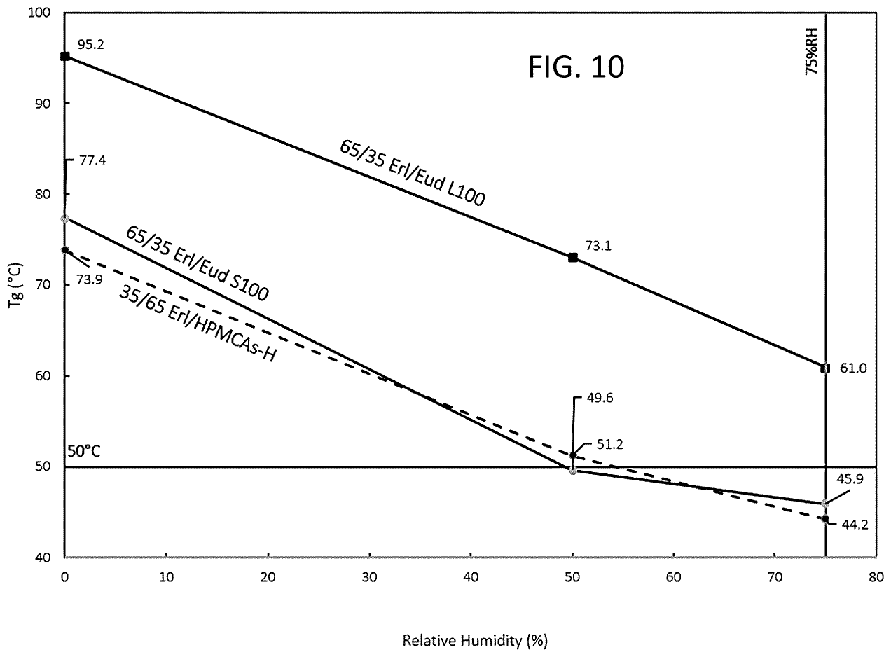

FIG. 10 is a graph showing the glass transition temperature (T_{g}) of the SADs as a function of relative humidity (RH). The results show that PMMAMA-based SADs prepared with Eudragit® L100 PMMAMA (having a 1:1 ratio of carboxyl to ester groups) have higher T_{g} values than SADs prepared with Eudragit® S100 PMMAMA (having a 1:2 ratio of carboxyl to ester groups) at all assessed RH conditions.

### Example 5

### High Loaded Dosage Forms (HLDF) with Posaconazole

Posaconazole is a rapid crystallizer with poor physical stability when included as the amorphous form in a SDF with a high drug loading. Dosages of posaconazole tablets are 300 mg/day, with an additional 300 mg loading dose on the first day, for prophylaxis of invasive Aspergillus and Candida infections in patients who are at high risk of developing these infections due to being severely immunocompromised, such as hematopoietic stem cell transplant (HSCT) recipients with graft-versus-host disease (GVHD) or those with hematologic malignancies with prolonged neutropenia from chemotherapy. Posaconazole has the following properties: Log P 4.5, pKa (base) 4.5, crystalline solubility in 0.5% simulated intestinal fluid (SIF) 2.2 g/mL, crystalline solubility in gastric buffer (GB) 33 g/mL, amorphous solubility in 0.5% SIF~55 g/mL, Tₘ 168° C., T_{g} 59° C., Tₘ/T_{g} (K/K) 1.3.

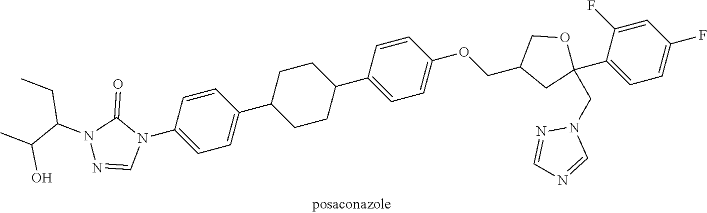 [SMILES: CCC(C(C)O)n1ncn(-c2ccc(C3CCC(c4ccc(OCC5COC(Cn6cncn6)(c6ccc(F)cc6F)C5)cc4)CC3)cc2)c1=O]

Spray solutions were prepared by dissolving the posaconazole and a dispersion polymer (PMMAMA or hydroxypropyl methylcellulose acetate succinate H grade) in 18/15 (w/w) dichloromethane/methanol at the desired ratio of posaconazole to polymer at a solids loading of 4%. Solutions were spray dried with an outlet temperature of 35-40° C. and an inlet temperature of 90-100° C. on a customized spray dryer (suitable for batch sizes from 0.5-200 grams) capable of drying gas flow rates of up to 35 kg/hr using a pressure swirl Schlick 2.0 spray nozzle (Düsen-Schlick GmbH, Untersiemau, Germany). After the spray drying process, spray dried dispersions were placed in a Gruenberg Benchtop Lab Dryer (Thermal Product Solutions, New Columbia, PA) for >18 hr at 30-35° C. to remove residual solvent.

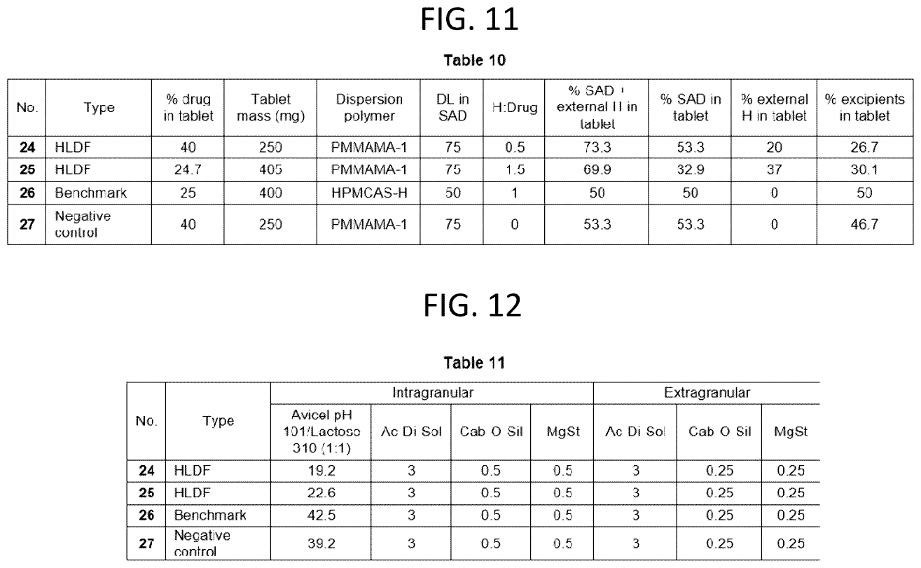

Tablet compositions 24-27 including 100 mg posaconazole were prepared as shown in Table 10 (FIG. 11), where SAD=spray-dried solid amorphous dispersion, DL=drug loading, H=HPMCAS-HF, and “external H” refers to HPMCAS-HF that is external to the SAD. The tablets included excipients as shown in Table 11 (FIG. 12). The excipients were a 1:1 blend of Avicel® PH-101 microcrystalline cellulose (a filler, available from DuPont Nutrition & Health) and Lactose 310 (a filler, available from UPI Chem., Somerset, NJ)), Ac-Di-Sol (croscarmellose sodium, a disintegrant, available from DuPont Nutrition & Health) Cab-O-Sil® fumed silica (a filler, available from Cabot Corporation, Alpharetta, GA), and magnesium stearate (MgSt; a lubricant).

The tablet compositions were made by preparing an intragranular (IG) blend of (i) a spray-dried SAD comprising posaconazole and a dispersion polymer (PMMAMA (i.e., Eudragit® L100 polymer, hereinafter “PMMAMA-1”) or HPMCAS-H) as indicated in Table 10 (FIG. 11), (ii) HPMCAS-HF (except for compositions 3 and 4), and (iii) IG excipients as indicated in Table 11 (FIG. 12). The IG blend was then blended with extragranular (EG) excipients as shown in Table 2 and compressed to form a tablet.

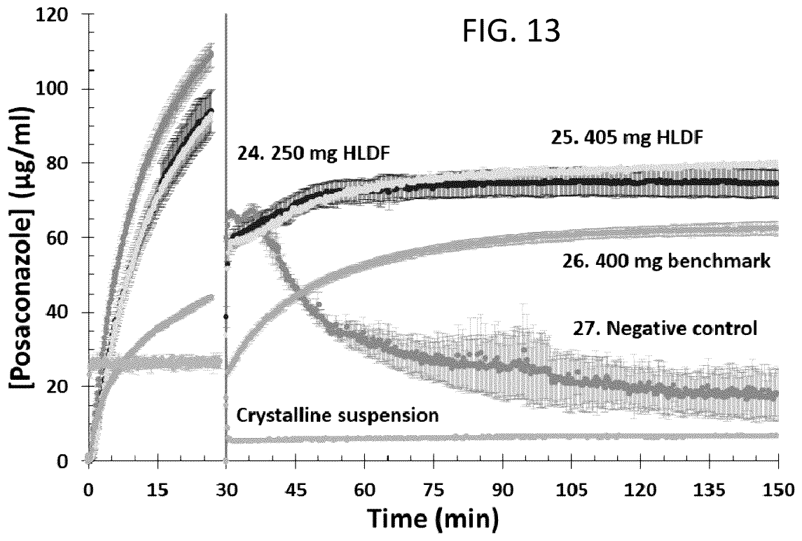

The tablet compositions were evaluated for dissolution performance and disintegration time (in 0.01 N HCl) as described in the Methods. The in vitro dissolution profiles of posaconazole tablets were compared to the commercially available crystalline posaconazole suspension, Noxafil® (40 mg per ml, Merck & Co., Inc.) as an additional negative control. To achieve a 100 mg dose of posaconazole, 2.5 ml of the Noxafil suspension were added to the dissolution vessel. The results are shown in Table 12 and FIG. 13.

TABLE 12

| | | AUC to 120 mins. | Disintegration Times |
|---|---|---|---|
| | | (μg*min/mL)*100 | (h:min:s) |
| 27 | Negative control | 29-42 | 0:00:22 |
| 24 | HLDF (0.5:1 H:Drug) | 83-91 | 0:00:51 |
| 25 | HLDF (1.5:1 H:Drug) | 89-90 | 0:01:16 |
| 26 | Benchmark | 65-68 | 0:00:34 |
| — | Noxafil ® suspension | 07-08 | |

### Example 6

### Physical Stability of Spray-Dried Dispersions with Posaconazole and PMMAMA-1 or HPMCAS-H

Spray-dried dispersions including different drug loadings (posaconazole) and a dispersion polymer—HPMCAS-H or PMMAMA-1—were prepared and subjected to accelerated physical stability studies as described in Methods. Drug loadings ranged from 50-85 wt % in PMMAMA-1 and 35-75 wt % in HPMCAS-H. In the stability studies, the SADs were placed in open containers inside a chamber set to a specified temperature and relative humidity.

Samples of the SDDs were removed from the chambers at 0, 1, 2, and 4 weeks and evaluated via:
Differential scanning calorimetry (DSC) to measure the glass transition temperature (T_{g}) and potential crystallization or melting events;
Powder x-ray diffraction (PXRD) to measure the presence of crystallinity (down to ~3% of sample mass); and
Scanning electron microscopy (SEM) to detect visual changes in morphology, fusing of SADs, and/or the presence of crystals.

A summary of the results is presented in Table 13, where DL=drug loading and RH=relative humidity. Examples 30-32 are benchmark compositions that do not include PMMAMA.

TABLE 13

| | Dispersion | % DL | | |
|---|---|---|---|---|
| | polymer | in SAD | Conditions | Results |
| 27 | PMMAMA-1 | 50 | 50° C., 75% RH | Stable (no change) |
| 28 | PMMAMA-1 | 75 | 50° C., 75% RH | Stable (no change) |
| 29 | PMMAMA-1 | 85 | 50° C., 75% RH | Stable (no change) |
| 30 | HPMCAS-H | 35 | 50° C., 75% RH | Stable (no change) |
| 31 | HPMCAS-H | 50 | 50° C., 75% RH | Stable (minimal |
| | | | | particle aggregation |
| | | | | observed at 4 weeks) |
| 32 | HPMCAS-H | 75 | 50° C., 75% RH | Unstable (particle |
| | | | | fusion and crystals |
| | | | | after 1 week) |

The results show that spray-dried SADs comprising PMMAMA-1 remained stable (i.e., the drug remained amorphous) for at least 4 weeks at drug loadings up to at least 85 wt %. Benchmark SADs comprising HPMCAS-H remained stable for at least 4 weeks at drug loadings up to 50 wt %. However, at a drug loading of 50 wt %, the benchmark SAD showed minimal particle aggregation after 4 weeks at the study conditions. At a drug loading of 75 wt %, the benchmark SAD showed particle fusion and crystals after one week at the study conditions. Thus, PMMAMA provided superior stability at higher drug loadings than the benchmark dispersion polymer HPMCAS-H.

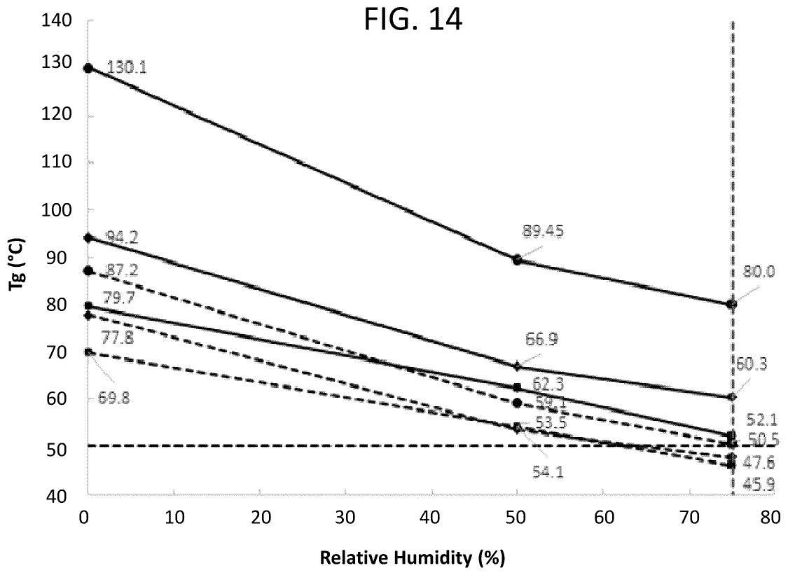

FIG. 14 is a graph showing the glass transition temperature T_{g} of the SADs as a function of relative humidity (RH); EUD L=Eudragit® L100 PMMAMA polymer. The T_{g} of Eudragit® L100 PMMAMA is 191° C.; the T_{g} of HPMCAS-H is 121° C. The results show that, at a given drug loading and % RH, PMMAMA-based SADs have higher T_{g} values than HPMCAS-H-based SADs. The results also show that HPMCAS-H-based SADs with 50 wt % (composition 31) and 75 wt % (composition 32) drug loadings have T_{g} values less than the accelerated stability storage temperature (50° C.) when the RH is 75%, which explains the poor stability of these SADs. In contrast, the Eudragit® L100 PMMAMA-based SADs (compositions 27, 28, 29) all have T_{g} values greater than the accelerated stability storage temperature (50° C.) at 75% RH, providing the PMMAMA-based SADs with greater storage stability.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An oral pharmaceutical composition comprising a solid dosage form (SDF), the SDF comprising:
at least 50 wt % of a solid amorphous dispersion (SAD) comprising a poorly water soluble active agent and a matrix material comprising poly[(methyl methacrylate)-co-(methacrylic acid)](PMMAMA), the PMMAMA having a glass transition temperature T_{g}≥135° C. at <5% relative humidity as measured by differential scanning calorimetry; and
at least 10 wt % of a concentration-sustaining polymer (CSP) comprising hydroxypropyl methylcellulose acetate succinate (HPMCAS), hydroxypropyl methylcellulose (HPMC), poly(vinylpyrrolidone-co-vinyl acetate) (PVPVA), carboxymethyl ethylcellulose (CMEC), or a combination thereof, and wherein the CSP is devoid of PMMAMA,
a poorly water soluble active agent dispersed in the SAD, wherein the active agent is at least 60 wt % of the SAD;
wherein the CSP is not dispersed in the SAD, and
wherein the SDF is a compressed tablet or caplet and wherein the compressed tablet or caplet comprises a compressed blend of the SAD and the CSP.

2. The oral pharmaceutical composition of claim 1, wherein the poorly water soluble active agent has a melting temperature Tₘ to glass transition temperature T_{g} ratio (K/K)≥1.3 and a Log P, where 1≤Log P≤10.

3. The oral pharmaceutical composition of claim 1, wherein the PMMAMA has a free carboxyl group to ester group ratio of from 1:0.8 to 1:2.2.

4. The oral pharmaceutical composition of claim 1, wherein at least 95% of particles of the SAD have an aspect ratio <10.

5. The oral pharmaceutical composition of claim 1, wherein the SAD further comprises at least one excipient.

6. The oral pharmaceutical composition of claim 1, wherein the SDF comprises:
a granular blend comprising particles of the SAD and particles of the CSP; or
an intragranular blend wherein individual granules comprise SAD particles and CSP particles.

7. The oral pharmaceutical composition of claim 6, wherein the SDF comprises an intragranular blend and at least some of the individual granules of the intragranular blend comprise SAD particles, CSP particles, and one or more intragranular excipients.

8. The oral pharmaceutical composition of claim 6, wherein the SDF further comprises one or more extragranular excipients.

9. The oral pharmaceutical composition of claim 1, wherein:
the SAD has a poorly water-soluble active agent loading of 60 wt % to 75 wt %; and
the SAD is 50 wt % to 70 wt % of the SDF.

10. An oral pharmaceutical composition comprising a solid dosage form (SDF), the SDF comprising:
at least 50 wt % of a solid amorphous dispersion (SAD) comprising a poorly water soluble active agent and a matrix material comprising poly[(methyl methacrylate)-co-(methacrylic acid)](PMMAMA), the PMMAMA having a glass transition temperature T_{g}>135° C. at <5% relative humidity as measured by differential scanning calorimetry; and
at least 10 wt % of a concentration-sustaining polymer (CSP) comprising hydroxypropyl methylcellulose acetate succinate (HPMCAS), hydroxypropyl methylcellulose (HPMC), poly(vinylpyrrolidone-co-vinyl acetate) (PVPVA), carboxymethyl ethylcellulose (CMEC), or a combination thereof, and wherein the CSP is devoid of PMMAMA,
a poorly water soluble active agent dispersed in the SAD, wherein the active agent is at least 60 wt % of the SAD;
wherein the CSP is not dispersed in the SAD, and
wherein the SDF is a compressed tablet or caplet wherein the compressed tablet or caplet comprises compressed SAD particles and an outer coating comprising the CSP.

11. An oral pharmaceutical composition comprising a solid dosage form (SDF), the SDF comprising:
at least 50 wt % of a solid amorphous dispersion (SAD) comprising a poorly water soluble active agent and a matrix material comprising poly[(methyl methacrylate)-co-(methacrylic acid)](PMMAMA), the PMMAMA having a glass transition temperature T_{g}≥135° C. at <5% relative humidity as measured by differential scanning calorimetry; and
at least 10 wt % of a concentration-sustaining polymer (CSP) comprising hydroxypropyl methylcellulose acetate succinate (HPMCAS), hydroxypropyl methylcellulose (HPMC), poly(vinylpyrrolidone-co-vinyl acetate) (PVPVA), carboxymethyl ethylcellulose (CMEC), or a combination thereof, and wherein the CSP is devoid of PMMAMA,
a poorly water soluble active agent dispersed in the SAD, wherein the active agent is from 60 wt % to 85 wt % of the SAD;
wherein the CSP is not dispersed in the SAD, and
wherein the SDF is a compressed tablet or caplet and wherein the compressed tablet or caplet comprises a compressed blend of the SAD and the CSP.

12. A method for making a solid dosage form, the method comprising:
providing a spray solution comprising a poorly water soluble active agent, from 2-9 wt % PMMAMA, and a solvent;
atomizing the spray solution into a chamber to form droplets;
introducing a drying gas into the chamber to dry the droplets and form particles of a solid amorphous dispersion comprising the poorly water soluble active agent and PMMAMA; and
forming the solid dosage form by combining the particles of the solid amorphous dispersion with a concentration-sustaining polymer in amounts suitable to provide at least 50 wt % of the solid amorphous dispersion and at least 10 wt % of the concentration-sustaining polymer in the solid dosage form.
